(19) United States
(12) Patent Application Publication
(10) Pub. No.: US 2018/0243000 A1
(43) Pub. Date: Aug. 30, 2018

(54) ECHOGENIC NEEDLES AND METHODS FOR MANUFACTURING ECHOGENIC NEEDLES

(71) Applicant: Injectimed, Inc., Minden, NV (US)

(72) Inventor: Thomas C. Kuracina, Carson City, NV (US)

(21) Appl. No.: 15/949,321

(22) Filed: Apr. 10, 2018

Related U.S. Application Data
(62) Division of application No. 15/017,867, filed on Feb. 8, 2016.

(51) Int. Cl. A61B 17/34; A61B 8/08; A61B 17/00

(52) U.S. Cl. CPC A61B 17/3403; A61B 2017/00526; A61B 2017/3413; A61B 8/0841

(57) ABSTRACT

A needle that includes an elongate tube having a proximal end, a distal end, an outer wall and an inner wall that defines a through passage, the through passage having a longitudinal central axis. The distal end of the tube includes a bevel having a length and that terminates at a distal tip. The inner wall of the tube has an exposed area in the bevel that is viewable from the top side of the tube. A spiral channel is formed in the inner wall of the tube along at least a portion of the length of the exposed area, the spiral channel having a proximal facing surface configured to reflect ultrasonic waves directed at it.

### FIELD OF THE INVENTION

The present invention provides medical needles with increased visibility in ultrasound guided procedures.

### BACKGROUND

Medical needles are used in clinical procedures to pierce tissue in order to subcutaneously deliver or infuse fluids and pharmaceutical compounds, collect blood, or percutaneously access the vascular or gastro-intestinal system to advance a guidewire to a target site in the body. Medical needles are used also to gather tissues, cells and/or bodily fluid for diagnostic purposes. In certain procedures, it is desirable to insert the needle tip to a specific location. For example, in some anesthesia procedures, anesthetic may need to be delivered beside a nerve, or a sensor placed in the vascular system to monitor the patient during surgery.

Proper placement of the needle and needle tip is crucial to insure a needle related injury does not occur to the patient. In certain procedures it is particularly important to avoid improperly piercing or damaging nerves, veins, arteries, and other organs or tissue during insertion of the needle. A double venipuncture, where the needle pierces one side of a blood vessel and is mistakenly advanced through the opposite side of the vessel, causes blood loss though the second puncture site that increases the probability of infection to the patient after the procedure is completed. For these reasons, clinicians use ultrasound, magnetic resonance or other imaging techniques for identifying the location of a probe, stylet, guidewire, tube or needle in the body. To this end, such devices have been provided with ultrasonic reflectors positioned on or within the circumferential exterior surface of such devices. A problem with such devices is that the roughened exterior surface of the needle can impede the smooth movement of the needle through the tissue and can cause discomfort to the patient. Another problem with previous echogenic needles is that they fail to provide for an unobstructed and clear visualization of the inside of the needle bevel.

### SUMMARY OF THE DISCLOSURE

According to some implementations medical needles, such as those used for surgical access, are provided that have one or more ultrasonic reflectors formed within an inner wall of the tube that forms the needle and positioned inside the exposed area of the bevel of the needle.

According to some implementations medical needles are provided that have one or more ultrasonic reflectors formed on or within the grind surfaces of the needle bevel.

According to some implementations medical needles are provided that have an ultrasonic reflector formed in the exposed area of the bevel at a location proximal to the bevel lancet.

According to some implementations medical needles are provided that have an ultrasonic reflector formed in the exposed area of the bevel lancet.

According to some implementations medical needles are provided that have an ultrasonic reflector that occupies all or a substantial portion of the exposed area of the bevel of the needle.

Methods of making echogenic needles are also provided.

### BRIEF DESCRIPTION OF THE DRAWINGS

FIGS. 1A-1C depict a conventional medical needle.

FIG. 2A is an isometric view of a medical needle having an ultrasonic reflector formed within the inner wall of the tube that forms the needle and positioned along the bottom of the exposed area of the bevel of the needle.

FIG. 2B is a cutaway frontal view of the medical needle of FIG. 2A with a partial cross-sectional portion showing the ultrasonic reflector.

FIG. 2C is a cross-sectional view of the ultrasonic reflector of FIG. 2A according to one implementation wherein the ultrasonic reflector comprises a radial surface.

FIG. 3 is an isometric view of a medical needle having an ultrasonic reflector formed within the inner wall of the tube that forms the needle and occupying all or a substantial portion of the exposed area of the bevel of the needle.

FIG. 4A is an isometric view of a medical needle having an ultrasonic reflector formed within the inner wall of the tube that forms the needle, the ultrasonic reflector being in the form of a longitudinal roughened surface that extends along at least a portion of the length of the exposed area in bevel of the needle.

FIG. 4B is a cutaway frontal view of the medical needle of FIG. 4A with a partial cross-sectional portion showing the ultrasonic reflector.

FIG. 5 is an isometric view of a medical needle having an ultrasonic reflector formed within the inner wall of the tube that forms the needle, the ultrasonic reflector being in the form of a roughened surface that occupies a substantial portion of the exposed area of the bevel of the needle.

FIG. 6A is an isometric view of a medical needle having an ultrasonic reflector formed within the inner wall of the tube that forms the needle, the ultrasonic reflector being in the form of a longitudinal channel that extends along at least a portion of the length of the exposed area of bevel of the needle.

FIG. 6B is a cutaway frontal view of the medical needle of FIG. 6A with a partial cross-sectional portion showing the ultrasonic reflector according to one implementation.

FIG. 6C is a cutaway frontal view of the medical needle of FIG. 6A with a partial cross-sectional portion showing the ultrasonic reflector according to another implementation.

FIG. 7 is an isometric view of a medical needle having an ultrasonic reflector formed within the inner wall of the tube that forms the needle, the ultrasonic reflector being in the form of a plurality of longitudinal channels that extends along at least a portion of the length of the exposed area of the bevel of the needle.

FIG. 8A is an isometric view of a medical needle having an ultrasonic reflector formed within the inner wall of the tube that forms the needle, the ultrasonic reflector being in the form of a spiral channel that extends along at least a portion of the length of the exposed area of the bevel of the needle.

FIG. 8B shows an enlarged top view of the bevel of the needle of FIG. 8A.

FIGS. 8C-8E show cutaway side views of the medical needle of FIGS. 8A and 8B with a partial cross-sectional portion showing the ultrasonic reflector according to various implementations.

FIG. 9A is an isometric view of a medical needle having an ultrasonic reflector formed within the inner wall of the tube that forms the needle, the ultrasonic reflector being in the form of one or more radially disposed channels located in the exposed area of the bevel of the needle.

FIG. 9B shows an enlarged top view of the bevel of the needle of FIG. 9A.

FIGS. 9C-9E show cutaway side views of the medical needle of FIGS. 9A and 9B with a partial cross-sectional portion showing the ultrasonic reflector according to various implementations.

FIG. 10A is an isometric view of a medical needle having an ultrasonic reflector in the form of an aperture that extends between the inner and outer walls of the tube that forms the needle and positioned along the bottom of the exposed area of the bevel of the needle.

FIG. 10B is a top view of a medical needle having an ultrasonic reflector in the form of an aperture with varying diameter that extends between the inner and outer walls of the tube that forms the needle and positioned along the bottom of the exposed area of the bevel of the needle.

FIG. 10C is a cross-sectional side view of a medical needle having an ultrasonic reflector in the form of a slanted aperture that extends between the inner and outer walls of the tube that forms the needle and positioned along the bottom of the exposed area of the bevel of the needle.

FIG. 11 is a side view of a medical needle according to any one of the implementations of FIGS. 2-10 located in a blood vessel, shown in cross section, at an oblique angle to an ultrasonic imaging system that emits ultrasonic waves toward the bevel of the needle and receives the ultrasonic waves reflecting off an ultrasonic reflector located in the exposed area of the bevel.

FIG. 12 is a side view of an extrusion tool forming one or more elongate channels in the inner wall of a tube to be formed into a needle.

FIG. 13 is a cross-sectional view of a tube formed with an extrusion tool according to one implementation.

FIG. 14 shows an apparatus and method for laser etching or laser drilling an ultrasonic reflector inside the bevel of a needle.

FIGS. 15A-15E illustrate an apparatus and method of manufacturing a needle having a spiral channel that extends along at least a portion of the length of the inside of the bevel of the needle.

FIGS. 16A-16E illustrate an apparatus and method of manufacturing a needle having one or more longitudinal channels that extend along at least a portion of the length of the inside of the bevel of the needle.

FIGS. 17A-17E illustrate an apparatus and method of manufacturing a needle having one or more longitudinal channels that extend along at least a portion of the length of the inside of the bevel of the needle and also a change in profile along an outer circumference of the needle tube proximal to the bevel.

FIGS. 18A-18E illustrate an apparatus and method of manufacturing a needle having one or more longitudinal channels that extend along at least a portion of the length of the inside of an enlarged bevel region of the needle.

FIGS. 19A-19E illustrate another apparatus and method of manufacturing a needle having one or more longitudinal channels that extend along at least a portion of the length of the inside of the bevel of the needle.

FIGS. 20A-20E illustrate an apparatus and method of manufacturing a needle having a longitudinal channel that extends along at least a portion of the length of the inside of the bevel of the needle.

FIG. 21A shows an enlarged side view of the channel forming apparatus of FIGS. 20A-20E with the blade in a non-cutting position.

FIG. 21B shows a top cross-section view of the channel forming apparatus of FIG. 21A along line A-A.

FIG. 21C shows an enlarged side view of the channel forming apparatus of FIGS. 20A-20E with the blade in a cutting position.

FIG. 21D shows a top cross-section view of the channel forming apparatus of FIG. 21C along line B-B.

FIG. 22A schematically shows a channel cutting apparatus having twelve cutting tools with a single blade on each cutting tool.

FIG. 22B schematically shows a channel cutting apparatus having twelve cutting tools with three blades on each cutting tool.

FIG. 23 is an isometric top view of a medical needle having an ultrasonic reflector formed on a grind surface of the bevel.

FIG. 24 is an isometric side view of a medical needle having an ultrasonic reflector formed in a grind surface of the bevel lancet.

FIGS. 25A and 25B respectively show isometric side and top views of a medical needle having an ultrasonic reflector formed on a grind surface of the bevel at a location proximal to the bevel lancet.

FIGS. 26A-26D illustrate a method of forming the ultrasonic reflector shown in FIGS. 25A and 25B.

FIG. 27 is a side view of an extrusion tool forming one or more elongate channels in an exterior wall of a tube to be formed into a needle.

FIG. 28A is a cross-sectional view of a tube formed with the extrusion tool of FIG. 27 according to one implementation.

FIG. 28B is a cross-sectional view of the tube of FIG. 28A located inside a catheter lumen.

FIGS. 29A and 29B show exemplary shapes of ultrasonic reflectors that may be formed by the use of the extrusion tool of FIG. 27.

FIG. 30A is an isometric view of a medical needle having an ultrasonic reflector in the form of an aperture with varying diameter that extends between the inner and outer walls of the tube at a location proximal to the bevel.

FIG. 30B is a top view of the needle of FIG. 30A.

FIG. 30C is a side view of the needle in FIG. 30A.

FIG. 31 is a cross-sectional side view of a medical needle having an ultrasonic reflector in the form of a slanted aperture that extends between the inner and outer walls of the tube that forms the needle at a location proximal to the bevel.

FIG. 32 is a cross-sectional side view of an epidural needle that includes an ultrasonic reflector on the inner wall of the needle near the distal end of the needle.

### DETAILED DESCRIPTION

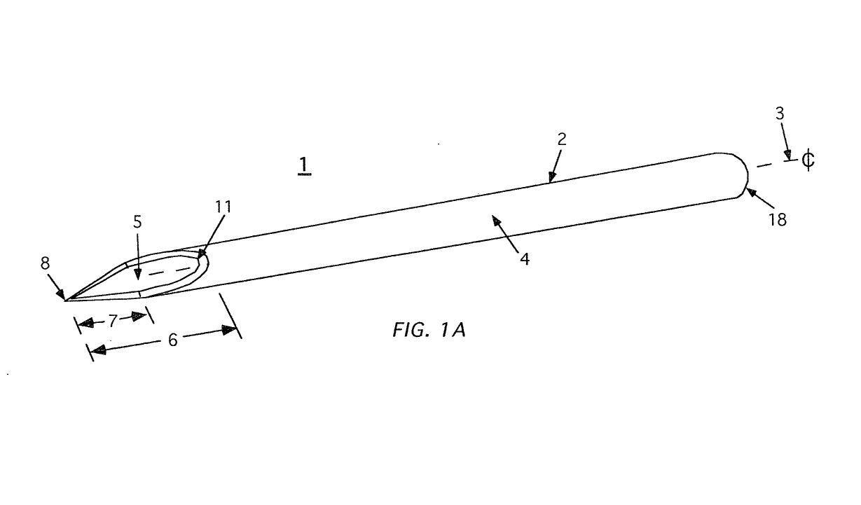

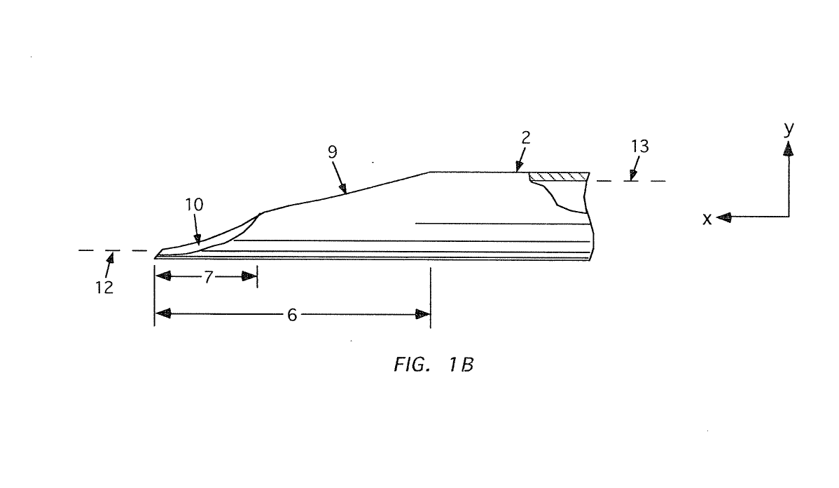

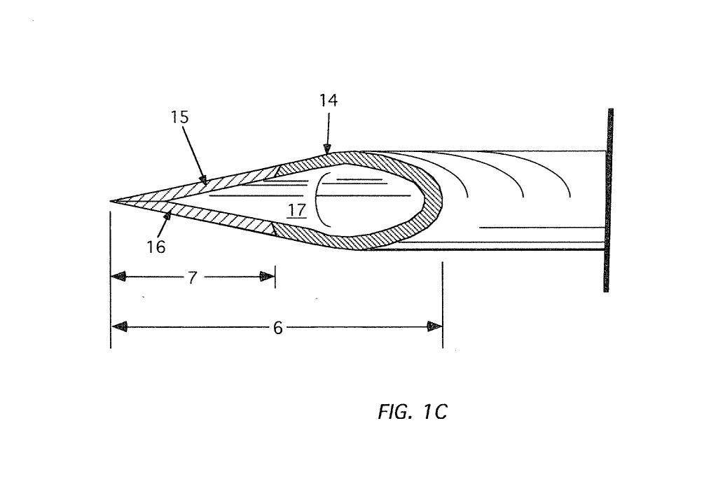

FIGS. 1A-1C depict a conventional medical needle 1. The needle 1 includes an elongate tube 2 having a proximal end 18, a central longitudinal axis 3, an outer wall 4 and an inner wall 5. The distal end of the needle includes a bevel 6 through which a portion of the inner wall 5 of the tube 2 is exposed. The bevel 6 includes a distal lancet 7 that converges into a tip 8 at the distal-most end of the needle. The bevel 6 may also include an anti-coring heel 11 located at a proximal end of the bevel. The bottom of the inner wall 5 of the tube 2 is co-extensive to the line 12 shown in FIG. 1B. The top of the inner wall 5 of the tube is co-extensive to the line 13 shown in FIG. 1B which is oriented 180 degrees from line 12.

The shape of the bevel 6 is generally produced by first grinding the distal end of the tube 2 to produce a sloped profile 9 as shown in FIG. 1B that runs from the proximal end of the bevel to the distal end of the tube 2. To create the lancet 7, a second set of grindings is generally made on the tube 2 to further reduce the tube profile to a point at tip 8. This may be accomplished, for example, by producing on each side of the lancet 7 a sloped profile 10 (as shown in FIG. 1B) that is distally co-extensive to and different than the sloped profile 9. This type of grinding process is illustrated in FIGS. 26B and 26C, and will be discussed in more detail below. FIG. 1C depicts a top view of the needle 1 of the top side of the bevel 6. Reference numeral 14 identifies a first grind surface produced during the first grinding of the tube 2. Reference numerals 15 and 16 identify second grind surfaces produced during the second set of grindings of the tube 2. Reference numeral 17 identifies the exposed area of the inner wall 5 of the tube 2 within the longitudinal length of the bevel 6.

Unless otherwise specified, the reference numerals and nomenclature used to identify the various parts of the needle 1 of FIGS. 1A-1C shall apply to the needles subsequently disclosed herein. Furthermore, the x and y coordinates hereinafter referred to correspond to the coordinates depicted in FIG. 1B, whereby the y axis is oriented orthogonal to the central axis 3 of the tube 2.

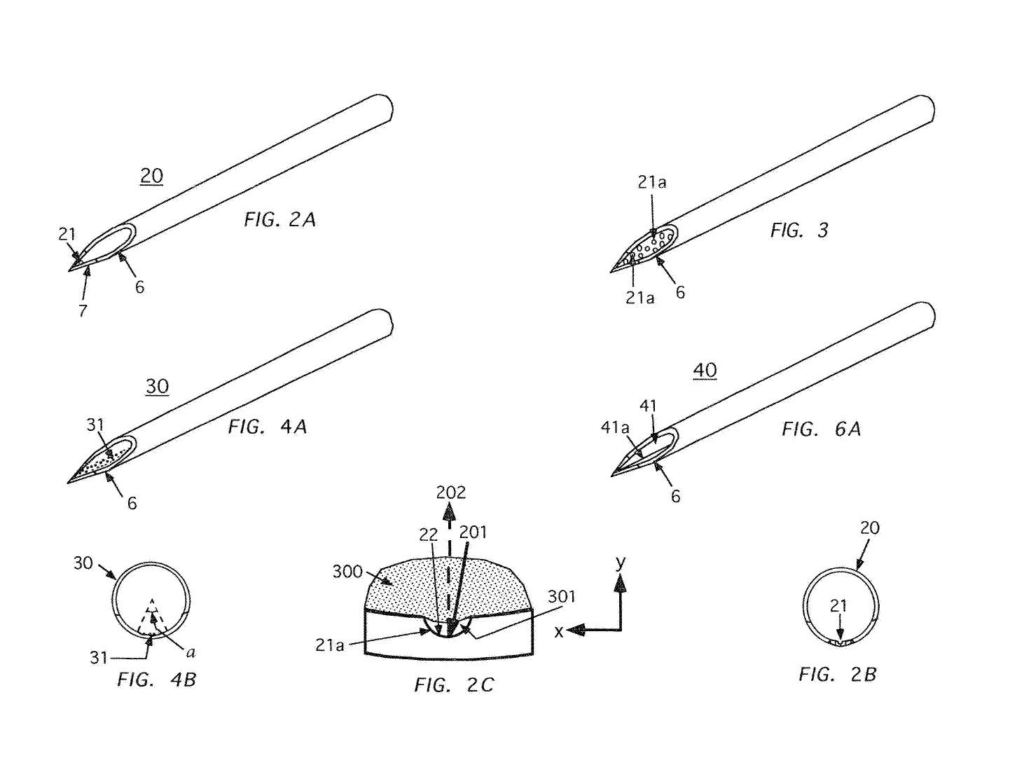

FIGS. 2-10 illustrate medical needles, such as those used for surgical access, having one or more ultrasonic reflectors formed within the exposed area 17 of the inner wall 5 of the tube 2 that forms the needle. As a result of the ultrasonic reflectors being located in the exposed area 17 of the bevel 6, clinicians are capable of more accurately positioning and angularly orientating the needle bevel within the patient through the use of an ultrasonic imaging system.

FIG. 2A is an isometric view of a medical needle 20 having an ultrasonic reflector 21 located within the exposed area 17 of the interior wall 5 of the tube 2 that forms the needle. FIG. 2B is a cutaway frontal view of the medical needle of FIG. 2A with a partial cross-sectional portion showing the ultrasonic reflector 21. The ultrasonic reflector 21 comprises a cavity 21a having at least one surface 22 facing toward the proximal end 18 of the tube 2 and oriented oblique to the y axis. The ultrasonic reflector 21 may be located within any portion of the exposed area 17 that allows for the unobstructed delivery and reflection of ultrasonic waves to and away from the ultrasonic reflector. The term “unobstructed” meaning that no portion of the needle itself stands in the way of the ultrasonic waves as they are being delivered to and reflected from the ultrasonic reflector.

By way of example, FIG. 11 illustrates an ultrasonic imaging system in the form of an ultrasonic transceiver 200 that is capable of both emitting ultrasonic waves 201 into the body 300 of a patient toward the bevel 6 of the needle and receiving ultrasonic waves 202 reflected back toward the transceiver by a ultrasonic reflector located within the bevel region. According to many of the implementations disclosed herein, ultrasonic reflectors are provided in the exposed area 17 of the bevel 6 and/or in a grind surface 14, 15, 16 of the bevel 6 in a manner that enables ultrasonic waves to be delivered to and reflected from the ultrasonic reflectors in an unobstructed manner as shown in FIG. 11.

As is apparent in FIG. 11, the angular orientation of the needle with respect to the transceiver 200 may vary dramatically among different medical procedures. For this reason, it is advantageous that the ultrasonic reflector comprise a surface or a variety of surfaces that are in the line of sight of the transceiver so as to be capable of reflecting ultrasonic waves 202 back to the transceiver 200 amongst a variety of angular orientations of the needle bevel 6. In furtherance of this objective, and with continued reference to FIGS. 2A-2C, according to one implementation the ultrasonic reflector 21 comprises a radial surface 22, as shown in FIG. 1C, that enables ultrasonic waves to be delivered to and reflected from the reflector 21 among multiple angular orientations of the bevel 6.

The ultrasonic reflector 21 may be produced in a number of ways, such as, for example, by a mechanical stamping or machining process, an electrical discharge machining process, laser etching, chemical etching, etc. According to one implementation the ultrasonic reflector 21 is located centered at the bottom of the exposed area 17 in the lancet 7 of the bevel 6. It is appreciated, however, that the reflector 21 may be located in other locations within the exposed area 17. For example, a single cavity 21a may be centrally located in the exposed area 17, or a plurality of cavities 21a may be dispersed centrally along the length of the bevel 6 and/or dispersed radially about the inner circumference of the bevel 6.

According to other implementations the ultrasonic reflector 21 may comprise a plurality of cavities 21a dispersed uniformly or randomly about all or a substantial portion of the exposed area 17 of the bevel as shown in FIG. 3. In such implementations the cavities 21a may be substantially the same or may comprise different shapes and sizes. The use of cavities with different shapes and sizes can increase the effectiveness of the ultrasonic reflector by providing a diversity of reflecting surfaces that are capable of reflecting ultrasonic waves back to a transceiver through a wide range of needle orientations.

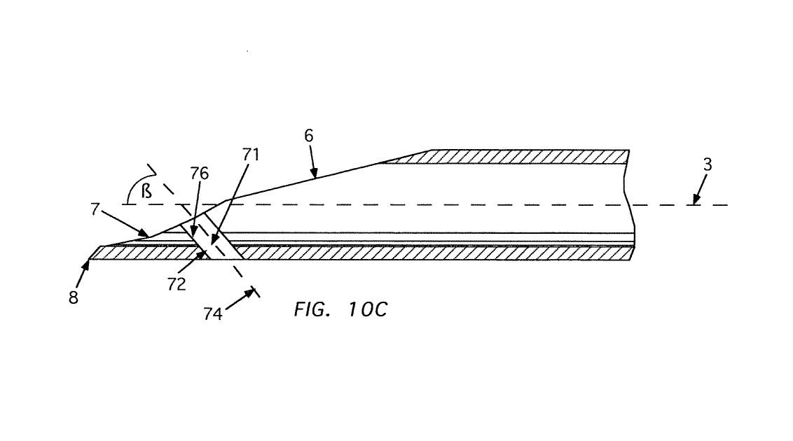

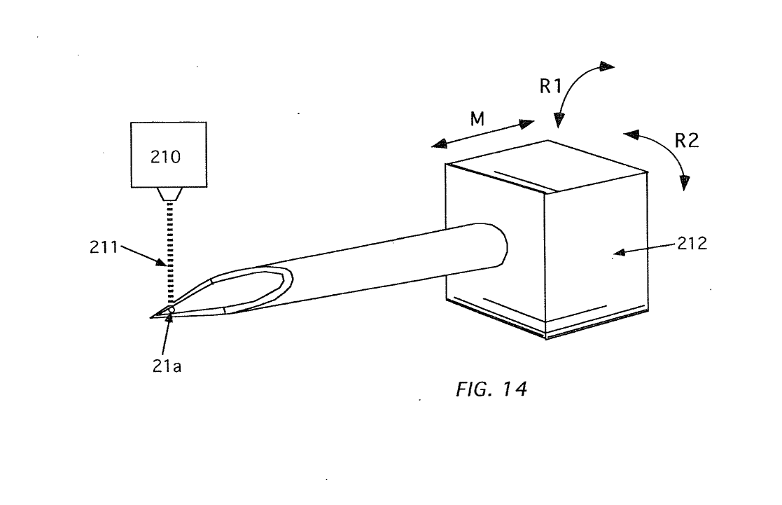

FIG. 14 shows a method of forming a cavity 21a by use of a laser source 210. The cavity 21a is created by precisely focusing a laser beam 211 at a target location of the exposed area 17 of the bevel 6. To create a single cavity 21a, as shown in FIGS. 1A-1C, the laser beam 211 and needle 10 may each be held stationary during the etching process. To create ultrasonic reflectors comprising multiple cavities (or channels as will be discussed in detail below), one or both the of the laser source 210 and needle may be moved relative to one another. For example, according to one implementation the tube 2 is supported in a fixture that moves (M) and/or rotates (R1 and R2) relative to a stationary laser source 210 as shown in FIG. 14. According to some implementations the laser source 210 may also move and rotate in the same manner.

In the implementation of FIG. 4A, an ultrasonic reflector 31 is formed within the inner wall 5 of the tube 2 that forms the needle 30, the ultrasonic reflector 31 being in the form of a longitudinal roughened surface that extends along at least a portion of the length of the exposed area 17 in the bevel 6 of the needle 30. FIG. 4B is a cutaway frontal view of the needle 30 with a partial cross-sectional portion showing the ultrasonic reflector 31. The ultrasonic reflector 31 may be produced in a number of ways, such as, for example, by bead blasting, laser etching, chemical etching. an electrical discharge machining process, laser chemical etching, etc. The roughened surface advantageously provides a large number of surface indentions of different sizes and shapes. As noted above, this can increase the effectiveness of the ultrasonic reflector by providing a diversity of reflecting surfaces that are capable of reflecting ultrasonic waves back to a transceiver through a wide range of needle orientations. According to one implementation only a narrow band of the length of the exposed area 17 of the bevel 6 is provided with the roughened surface as shown in FIGS. 4A and 4B. Such a configuration enables a clinician to more accurately orient the rotational position of the bevel 6 since visualization of the bevel will occur within a narrow rotational band. According to some implementations the radial width of the band is in the form of an arc that spans an angle a (as shown in FIG. 4B) of between 10 to 180 degrees, preferably between 10 and 90 degrees, and more preferably between 10 and 45 degrees.

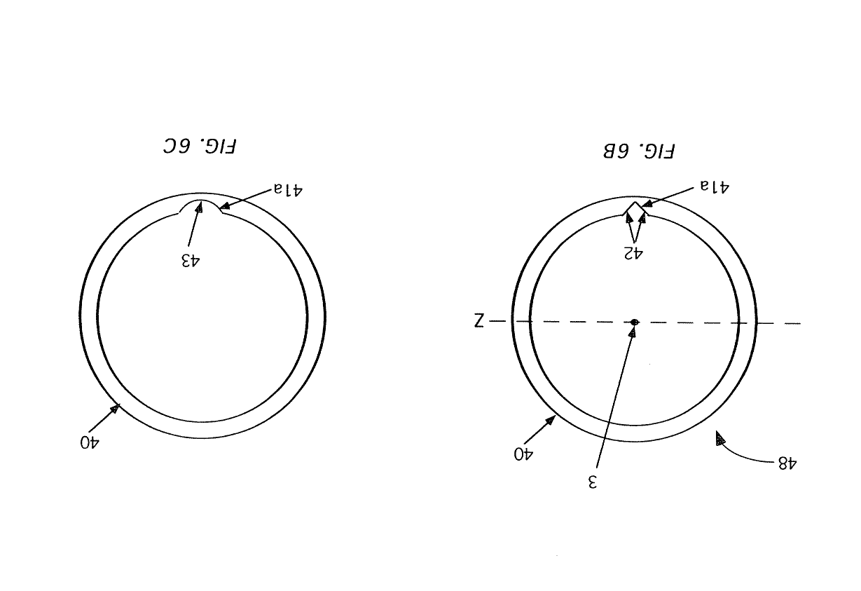

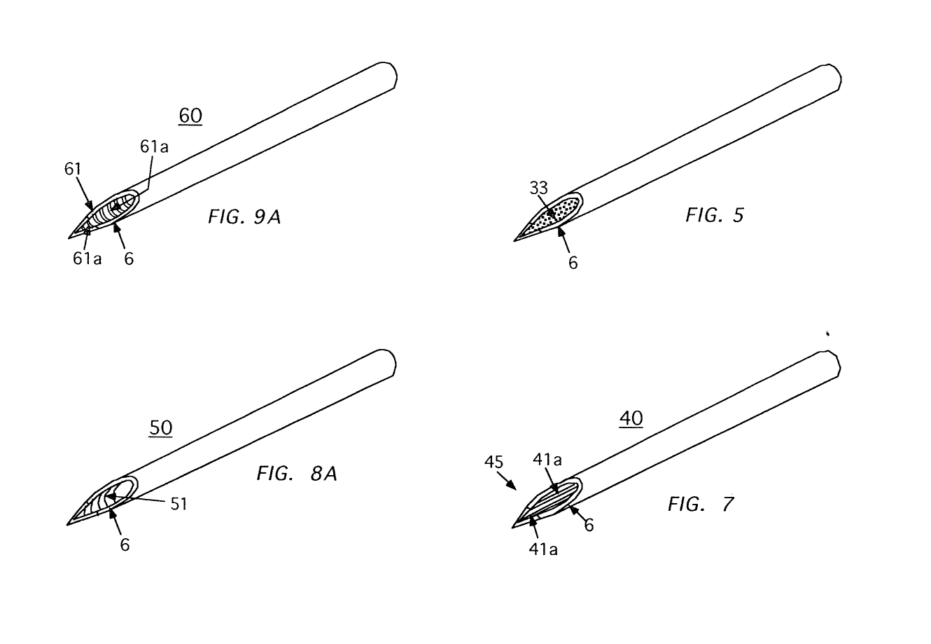

According to other implementations, as shown in FIG. 5, an ultrasonic reflector 33 is provided that comprises a roughened surface that occupies all or a substantial portion of the exposed surface 17 of the bevel 6.

In the needle 40 of FIG. 6A an ultrasonic reflector 41 is formed within the inner wall 5 of the tube 2 that forms the needle, the ultrasonic reflector 41 being in the form of a longitudinal channel 41a having a depth and that extends along at least a portion of the length of the exposed area 17 in the bevel 6 of the needle. As shown in FIG. 6A the bevel 6 contains a single channel 41a that runs parallel to the needle axis 3 down the bottom and center of the exposed area 17. According to one implementation the channel 41a has a V-shape with converging facing side walls 42 that are capable of producing reflected ultrasonic waves directed toward an ultrasonic sensor position above the top side 48 of the needle 40 as shown in FIG. 6B. The top side of the needle being defined herein as that portion of the needle residing above the Z-plane as shown in FIG. 6B. According to another implementation the channel 41a has a curved shape with a curved wall 43 that is capable of producing reflected ultrasonic waves directed toward an ultrasonic sensor position above the top side of the needle 40 as shown in FIG. 6C. It is appreciated that the channel 41a may take any of a variety of shapes that provide at least one surface that is capable of reflecting ultrasonic waves toward an ultrasonic sensor positioned above the top side of the needle.

According to other implementations, as shown in FIG. 7, an ultrasonic reflector 45 is provided in the form a plurality of longitudinal, radially spaced-apart channels 41a arranged about the inner circumference of the exposed surface 17 in the bevel 6 of the needle 40.

Exemplary methods for forming channels in the bevel 6 of the needle are discussed in detail below.

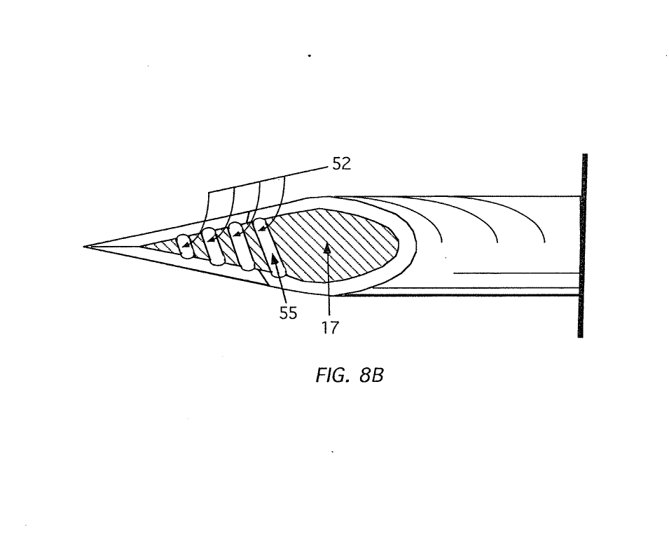

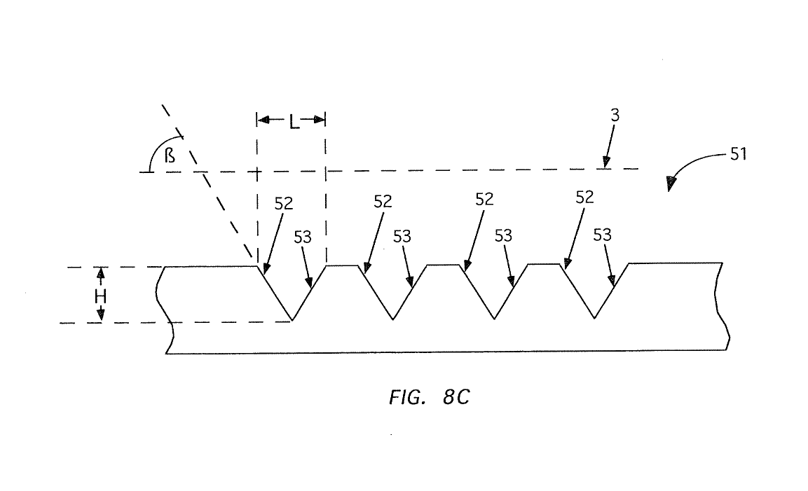

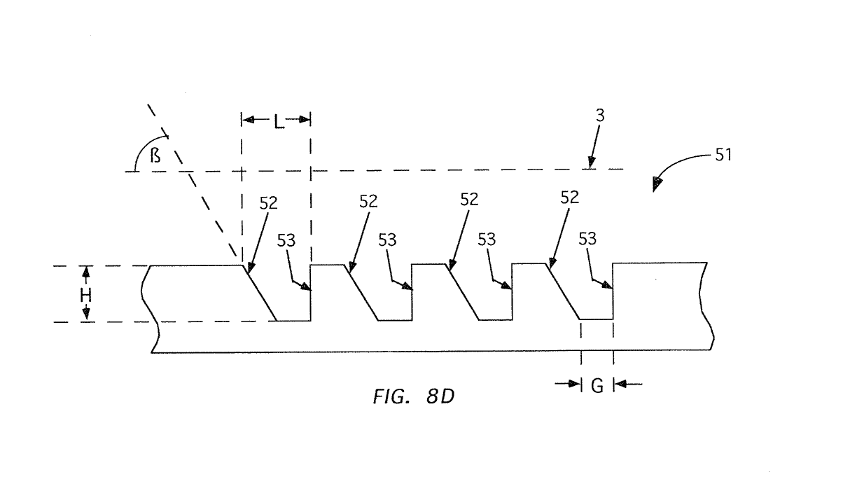

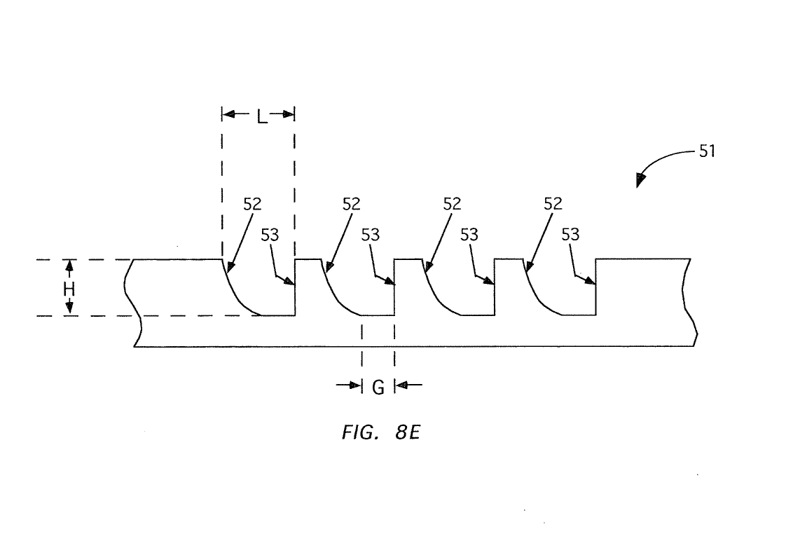

In the implementation of FIGS. 8A and 8B, a medical needle 50 is provided that has an ultrasonic reflector 51 formed within the inner wall 5 of the tube 2 that forms the needle, the ultrasonic reflector being in the form of a spiral channel 55 that extends along at least a portion of the length of the exposed area 17 in the bevel 6 of the needle. As shown in FIG. 8B, the spiral channel 55 includes proximal facing surfaces 52 on which ultrasonic waves may be reflected toward an ultrasonic sensor positioned above the top side of the needle. The needle 50 may be manufactured, for example, using the method shown in FIGS. 15A-15E, which is described in detail below. In the context of the implementations disclosed and contemplated herein, the spiral channel 55 in the bevel 6 of the needle is not continuous when the bevel 6 has been formed in the tube 2. Because these channels typically originate from a continuous spiral, as shown in the example of FIG. 15D, they will nonetheless being referred to as being spiral. It is appreciated, however, that the spiral oriented channels maybe formed in the inner wall 5 of the tube 2 after the formation of the bevel 6.

According to some implementations the spiral channel 55 extends only along a length of the lancet 7 portion of the bevel 6. In other implementations the spiral channel 55 spans all or substantially all of the length of the exposed area 17.

FIGS. 8C-8E show cutaway side views of the medical needle of FIG. 8A with a partial cross-sectional portion showing the ultrasonic reflector 51 according to various implementations.

In the implementation of FIG. 8C the spiral channel 55 has a V-shape cross-section having sloped proximal facing surfaces 52 and sloped distal facing surfaces 53. In the implementation shown in FIG. 8C the proximal facing surfaces 52 are linear and are sloped at an angle β in relation to the central axis 3 of the needle in a range between 10 and 80 degrees, and preferably between 20 to 60 degrees. According to some implementations to provide a line-of-sight between the proximal facing surfaces 52 and an ultrasonic transceiver located above the top side of the needle for a wide range of angular orientations between the bevel 6 and the ultrasonic transceiver, the ratio of the length L of the upper channel opening to the height of the channel H is greater than 1.0. According to some implementations the L/R ratio is between 1.0 and 2.0. According to some implementations the spiral channel has a U-shaped cross-section (not shown) wherein each of the proximal and distal facing surfaces 52 and 53 is curvilinear.

In the implementation of FIG. 8D the spiral channel 55 comprises non-converging proximal and distal facing surfaces 52 and 53 that are separated at their base by a gap G. As with the implementation of FIG. 8C, the proximal facing surfaces 52 are linear and are sloped at an angle β in relation to the central axis 3 of the needle in a range between 10 and 80 degrees, and preferably between 20 to 60 degrees. According to some implementations to provide a line-of-sight between the proximal facing surfaces 52 and an ultrasonic transceiver located above the top side of the needle for a wide range of angular orientations between the bevel 6 and the ultrasonic transceiver, the ratio of the length L of the upper channel opening to the height of the channel H is greater than 1.0. According to some implementations the L/R ratio is between 1.0 and 2.0.

In the implementation of FIG. 8E the spiral channel 55 comprises non-converging proximal and distal facing surfaces 52 and 53 that are separated at their base by a gap G. The implementation of FIG. 8E differs from the implementation of FIG. 8D in that the proximal facing surfaces 52 are curvilinear instead of linear. Like the preceding implementations, to provide a line-of-sight between the proximal facing surfaces 52 and an ultrasonic transceiver located above the top side of the needle for a wide range of angular orientations between the bevel 6 and the ultrasonic transceiver, the ratio of the length L of the upper channel opening to the height of the channel H is greater than 1.0. According to some implementations the L/R ratio is between 1.0 and 2.0.

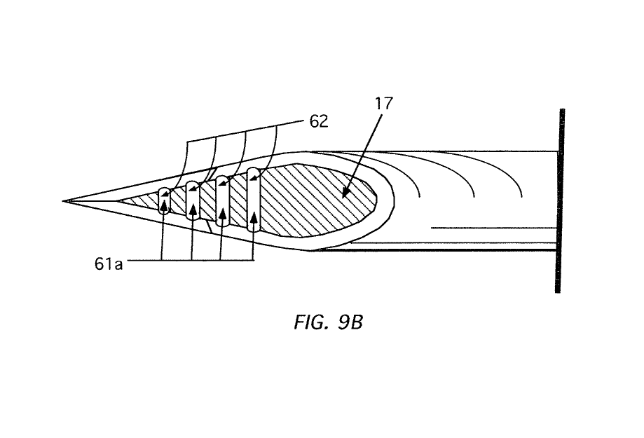

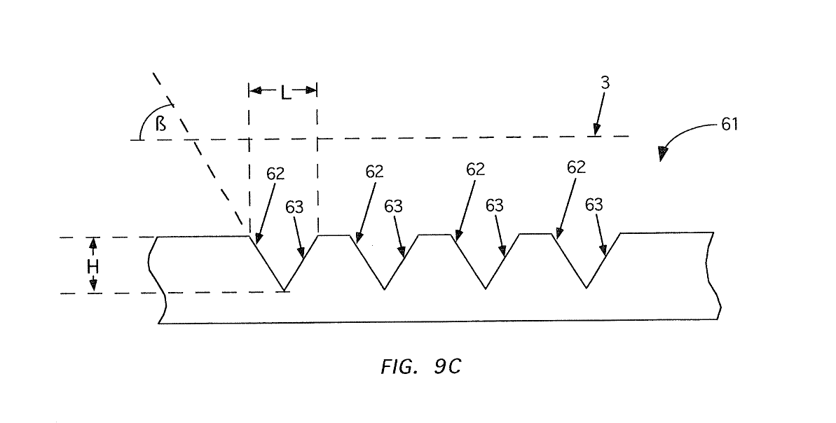

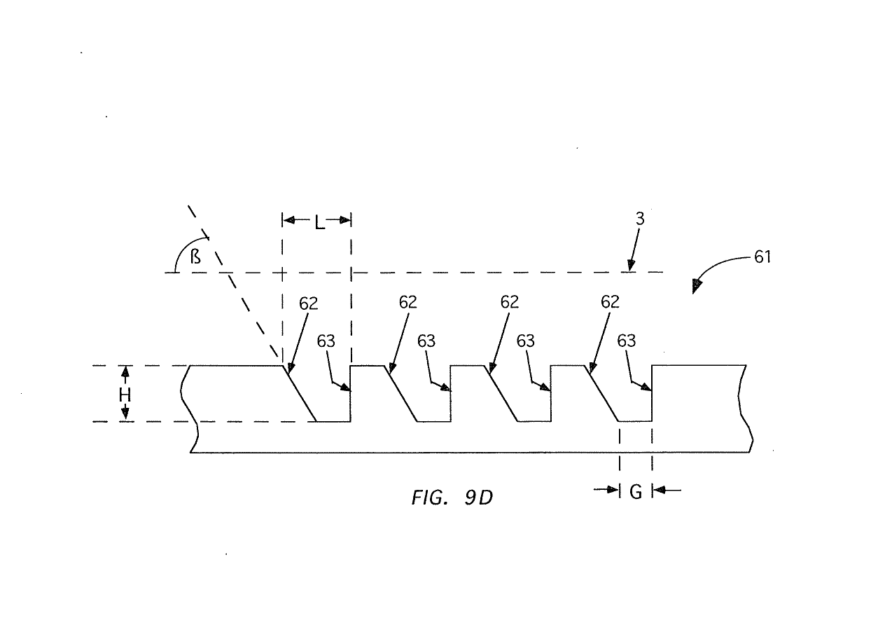

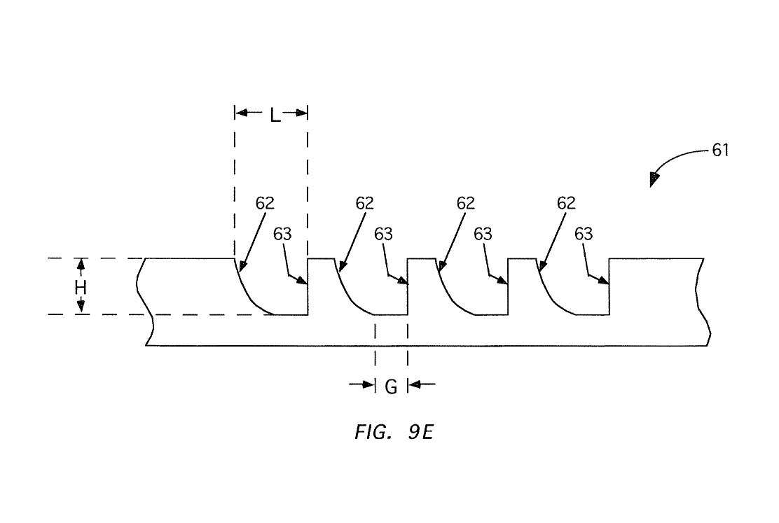

FIG. 9A is an isometric view of a medical needle 60 having an ultrasonic reflector 61 formed within the inner wall 5 of the tube 2 that forms the needle, the ultrasonic reflector 61 being in the form of one or more radial channels 61a located in the exposed area 17 of the bevel 6. FIG. 9B shows an enlarged top view of the bevel region of the needle of FIG. 9A. The needle 50 may be manufactured, for example, using the method shown in FIGS. 16A-16E, which is described in detail below. In the context of the implementations disclosed and contemplated herein, and as most clearly shown in FIG. 9B, the term radial channel denotes a channel that extends circumferentially about at least a portion of the inner diameter of the inner passage way of the tube 2. In various implementations disclosed herein, this occurs within the exposed area 17 of the bevel 6.

According to some implementations the one or more channels 61a are located only in the lancet 7 portion of the bevel 6.

As shown in FIG. 9B, each of the radial channels includes a proximal facing surfaces 62 on which ultrasonic waves may be reflected toward an ultrasonic sensor positioned above the top side of the needle. The implementation of FIGS. 9A and 9B differs from the implementation of FIGS. 8A and 8B in that the one or more channels 61a are not formed at an oblique angle to the circumference of the inner wall 5 of the needle tube 2.

In the implementation of FIG. 9C, the one or more channels have a V-shape cross-section having sloped proximal facing surfaces 62 and sloped distal facing surfaces 63. In the implementation shown in FIG. 9C the proximal facing surfaces 62 are linear and are sloped at an angle β in relation to the central axis 3 of the needle in a range between 10 and 80 degrees, and preferably between 20 to 60 degrees. According to some implementations, to provide a line-of-sight between the proximal facing surfaces 62 and an ultrasonic transceiver located above the top side of the needle for a wide range of angular orientations between the bevel 6 and the ultrasonic transceiver, the ratio of the length L of the upper channel opening to the height of the channel H is greater than 1.0. According to some implementations the L/R ratio is between 1.0 and 2.0. According to some implementations the spiral channel has a U-shaped cross-section (not shown) wherein each of the proximal and distal facing surfaces 62 and 63 is curvilinear.

In the implementation of FIG. 9D the one or more channels 61a each comprises non-converging proximal and distal facing surfaces 62 and 63 that are separated at their base by a gap G. As with the implementation of FIG. 9C, the proximal facing surfaces 62 are linear and are sloped at an angle β in relation to the central axis 3 of the needle in a range between 10 and 80 degrees, and preferably between 20 to 60 degrees. According to some implementations to provide a line-of-sight between the proximal facing surfaces 62 and an ultrasonic transceiver located above the top side of the needle for a wide range of angular orientations between the bevel 6 and the ultrasonic transceiver, the ratio of the length L of the upper channel opening to the height of the channel H is greater than 1.0. According to some implementations the L/R ratio is between 1.0 and 2.0.

In the implementation of FIG. 9E the one or more channels 61a each comprise non-converging proximal and distal facing surfaces 62 and 63 that are separated at their base by a gap G. The implementation of FIG. 9E differs from the implementation of FIG. 9D in that the proximal facing surfaces 62 are curvilinear and not linear. Like the preceding implementations, to provide a line-of-sight between the proximal facing surfaces 62 and an ultrasonic transceiver located above the top side of the needle for a wide range of angular orientations between the bevel 6 and the ultrasonic transceiver, the ratio of the length L of the upper channel opening to the height of the channel H is greater than 1.0. According to some implementations the L/R ratio is between 1.0 and 2.0.

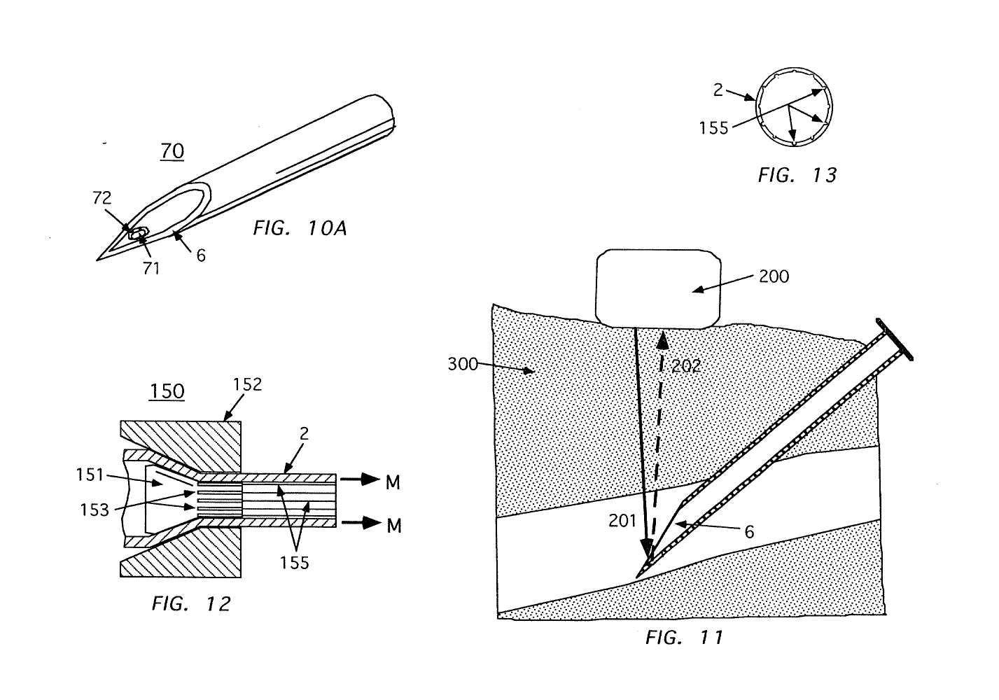

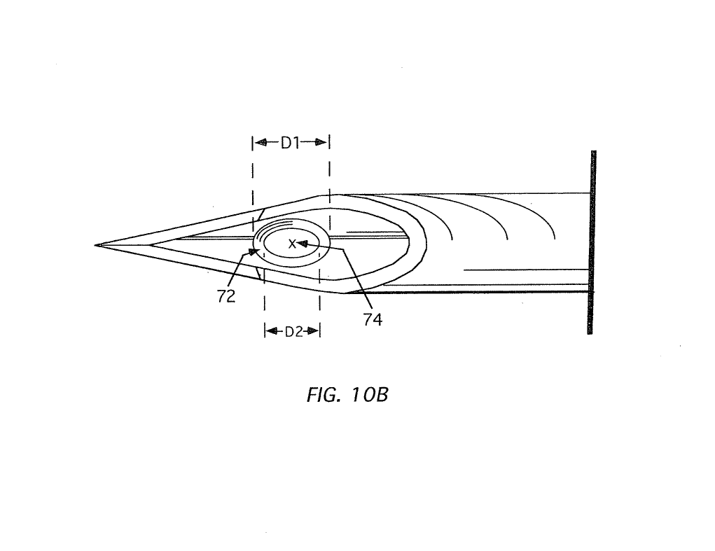

FIG. 10A is an isometric view of a medical needle 70 having an ultrasonic reflector 71 in the form an aperture that extends between the inner and outer walls 4 and 5 of the tube 2 that forms the needle and positioned along the bottom of the exposed area 17 of the bevel 6 of the needle 70. The inner circumferential wall 72 of the aperture 71 provides the surface for reflecting ultrasonic waves that are directed to it from an ultrasonic transceiver located above the top side of the needle. According to one implementation the ultrasonic reflector 71 is located centered at the bottom of the exposed area 17 in the lancet 7 of the bevel 6. It is appreciated, however, that the reflector 71 may be located in other locations within the exposed area 17. For example, a single through aperture may be centrally located in the exposed area 17, or a plurality of through apertures may be dispersed centrally along the length of the bevel 6 and/or dispersed radially about the inner circumference of the bevel 6.

FIG. 10B shows a top view of the through aperture 71 according to one implementation wherein the diameter D1 of the aperture 71 at the inner wall 5 of the tube 2 is greater than the diameter D2 of the aperture 71 at the outer wall 4 of the tube 2, with the inner circumferential wall 72 being sloped. This configuration has several advantages. First it provides a larger surface for reflecting ultrasonic waves directed toward the aperture from above the top side of the needle. This, in conjunction with, the sloped surface of the inner circumferential wall 72 enhances the ultrasonic reflector's ability to reflect ultrasonic waves back toward an ultrasonic transceiver located above the top side of the needle amongst a wider range of angular orientations of the needle bevel 6 in comparison to an aperture having a uniform diameter throughout its length.

In the implementations illustrated in FIGS. 10A and 10B the central axis 74 of the aperture 71 is substantially perpendicular to the longitudinal axis 3 of the needle tube 2. However, according to other implementations, as shown in FIG. 10C, a slanted aperture 71 is formed in the wall of the needle tube 2 so that the central axis 74 of the aperture 71 is not perpendicular to the longitudinal axis 3 of the tube 2. This configuration has several advantages. First it provides a larger exposed proximal facing surface 76 for reflecting distally directed ultrasonic waves. This, in conjunction with the sloped surface of the inner circumferential wall 72 enhances the ultrasonic reflector's ability to reflect ultrasonic waves back toward an ultrasonic transceiver located above the top side of the needle amongst a wider range of angular orientations of the needle bevel 6 in comparison to an aperture arranged orthogonal to the central axis of the needle with a uniform diameter throughout its length.

According to one method the oblique/slanted aperture is formed by a tool or laser that bores through the wall of the tube 2 at an angle. According to one implementation the tool or laser is aligned centered with the bottom 12 of the exposed area 17 of the bevel 6, facing in the proximal direction. The slanted aperture is then cut at a target location within the exposed area 17 of the bevel 6. According to one implementation the target location is in the lancet 7 of the bevel 6 as shown in FIG. 10D.

According to some implementations the centerline of the central axis of the slanted aperture 71 forms an angle β with the central axis 3 of the tube with the angle β being between 20 to 80 degrees, and preferably between 30 to 70 degrees. However, in any event the angle β is always less than 90 degrees.

As described above, ultrasonic reflectors in the form of one or more longitudinal channels may be formed in the inner wall 5 of the tube 2 at the bevel 6 of the needle to enhance the bevel's echogenicity. FIG. 12 shows a side view of an extrusion tool 150 that includes a mandrel 151 and a female die 152. In the implementation shown, the mandrel includes 12 cutting elements 153 dispersed equidistantly about the outer circumference of the mandrel. According to one implementation stainless steel tube stock is drawn through the extrusion tool 150 in the direction M in order to produce the needle tube 2. During the extrusion process the cutting elements 153 impinge on the inner wall of the tube to forming twelve longitudinal channels 155 that run the length of the tube. FIG. 13 is a cutaway frontal view of the tube 2 with a partial cross-sectional portion showing the twelve longitudinal channels 155. Upon having produced the tube 2 having the longitudinal channels 155, the distal end of the tube 2 is subjected to one or more grinding processes as described above to form the bevel 6 of the needle. According to some implementations the tube 2 is rotated as it is advanced across the mandrel 151 to produce one or more spiral channels in the inner wall 4 of the tube 2.

Turning again to FIG. 2C, it is noted that another benefit may result from the placement of ultrasonic reflectors within the exposed area 17 of the bevel 6. Ultrasonic waves are more strongly reflected at the interface of two materials, such as at the needle and tissue interface. This applies also to interfaces involving voids. In needles having anti-coring heels, the anti-coring heel tends to lift on the tissue into which the bevel has been inserted. This phenomenon, in conjunction with the structure of the recesses that form the ultrasonic reflector provided herein, can result in the tissue being suspended above at least a portion of the ultrasonic reflector as shown in FIG. 2C. As seen in FIG. 2C, at the ultrasonic reflector 21a this produces no less than two material interfaces. A first interface exists between the patient tissue 300 and the gas or fluid 301 residing inside the cavity 21a, and a second interface exists between the gas or fluid residing inside the cavity 21a and the reflecting surface 22 of the cavity 21a. Hence, placement of the ultrasonic reflectors in the bevel of the needle as provided herein can more strongly reflect ultrasonic waves in comparison to ultrasonic reflectors not located inside exposed area 17 of the bevel 6.

As previously explained, according to some implementations ultrasonic reflectors are provided in the form of a spiral channel that extends through at least a distal end of the tube 2, in the region of the bevel 6. FIGS. 15A-15E illustrate an apparatus and method for producing a spiral channel reflector according to one implementation. The apparatus includes a die 350 having a first end section 351 with a cavity 355 configured to receive the distal end 354 of the tube 2. The cavity 355 has a diameter D4 that is slightly greater than the external diameter D3 of the tube 2. The cavity 355 includes a ledge 356 at one end that is configured to support the distal end 354 of the tube 2. The die 350 also includes a second end section 352 that includes a cavity for receiving a first mandrel 370 having a spiral protruding cutting element 371. According to some implementations, the shape of the cutting element 371 is selected to produce channel geometries like those described above. The first mandrel 370 is moveable within die 350 and has an outer-most diameter D2 (defined by the cutting element 371) that is greater than the internal diameter D1 of the tube 2. The apparatus includes a second mandrel 360 that is inserted into a opening in the proximal end 353 of the tube 2 in the direction M1. The second mandrel 360 has an enlarged proximal head 361 having a flange surface 362 configured to rest on the distal end 353 of the tube 2. The length of the second mandrel 360 is less than the tube 2.

As shown in FIG. 15B, initially the first mandrel 370 is positioned within the second end section 352 of the die 350 and the distal end 354 of the tube is positioned within the cavity 355 of the die so that the distal end 354 rests on the ledge 356. The second mandrel 360 is also fully inserted into the tube 2 so that the flange surface 362 rests against the proximal end 353 of the tube 2. In this position a force F is applied to the end of the second mandrel 360 to hold the tube firmly within the cavity 355 of the die 350. As shown in FIG. 15B, at this stage the distal end portion of the tube 2 includes a void 376 for receiving the first mandrel 370. According to one implementation the length of the void 376 corresponds to the length of the bevel 6 to be created in the distal end portionof the tube 2 in a subsequent step, as discussed below. The spiral channel 380 is cut by rotating the first mandrel 370 counter-clockwise R1 (or clockwise) while advancing the end of the first mandrel 370 in the direction M2 into the distal end portion of the tube 2 designated to be formed into the bevel 6. The first mandrel 370 is thereafter extracted from the tube 2 by rotating the mandrel in the clockwise R2 (or counter-clockwise) as shown in FIG. 15C. The tube 2 is then removed from the apparatus as shown in FIG. 15D.

Upon having produced the tube 2 having the spiral channel 380, the distal end portion of the tube 2 is subjected to one or more grinding processes as described above to form the bevel 6 of the needle as shown in FIG. 15F.

In the apparatus shown in FIGS. 15A-15D the cutting element 371 of the first mandrel 371 is a spiral element. In other implementations the cutting element may include a discrete cutting element that is position at or near the end of the first mandrel. In such an implementation the spiral channel is formed by a controlled simultaneous rotation and advancement of the distal end of the first mandrel 370 into the distal end portion of the tube 2.

As discussed above, according to some implementations ultrasonic reflectors are provided in the form of one or more longitudinal channels that extend through at least a distal end of the tube 2, in the region of the bevel 6. FIGS. 16A-16E illustrate an apparatus and method for producing multiple longitudinal channels in the distal end of the tube 2 according to one implementation. The apparatus is similar to the apparatus of FIGS. 15A except that the first mandrel 370 includes a plurality of longitudinal cutting elements 372 that radially extend from the surface of the mandrel. In the implementation of FIG. 16, a plurality of longitudinal cutting elements is provided with the cutting elements being equidistantly spaced about the outer circumference of the first mandrel. In other implementations the cutting elements 372 circumscribe only a portion of the first mandrel 370 and are configured to act on the distal end portion of the tube 2 where the bevel 6 is intended to reside.

As shown in FIG. 16B, initially the first mandrel 370 is positioned within the second end section of the die 350 and the distal end 354 of the tube is positioned within the cavity 355 of the die so that the distal end rests on the ledge 356. The second mandrel 360 is also fully inserted into the tube 2 so that the flange surface 362 rests against the distal end 353 of the tube 2. In this position a force F is applied to the end of the second mandrel 360 to hold the tube firmly within the cavity 355 of the die 350. As shown in FIG. 16B, at this stage the distal end portion of the tube 2 includes a void 376 for receiving the first mandrel 370. The longitudinal channels 381 are cut by advancing the end of the first mandrel 370 in the direction M2 into the distal end portion of the tube 2. The first mandrel 370 is thereafter retracted from the tube 2 by moving the mandrel in the direction M3. The tube 2 is then removed from the apparatus as shown in FIG. 16D.

Upon having produced the tube 2 having the longitudinal channels 381, the distal end portion of the tube 2 is subjected to one or more grinding processes as described above to form the bevel 6 of the needle as shown in FIG. 16E.

FIGS. 17A-17E illustrate an apparatus and method of forming an ultrasonic reflector at the distal end of a tube 2, and to provide the tube 2 with an enlarged external profile 398 at a location proximal to the ultrasonic reflector. The apparatus is similar to that of FIG. 16A and includes a third mandrel 390. As shown in the figures, the tube 2 containing the second mandrel 360 slides within the third mandrel 390. The distal end of third mandrel 390 includes a circular cavity that has a diameter that is greater than the outer diameter of the tube 2.

As shown in FIG. 17B, initially the first mandrel 370 is positioned within the second end section of the die 350 and the distal end 354 of the tube is positioned within the cavity 355 of the die so that the distal end 354 rests on the ledge 356. The second mandrel 360 is also fully inserted into the tube 2 so that the flange surface 362 rests against the proximal end 353 of the tube 2. The tube 2 containing the second mandrel 360 is also fully inserted into the third mandrel 390. With the distal end 394 of the third mandrel 390 resting on the proximal end 359 of the die 350 forces F are applied to the second and third mandrels to maintain them in their respective positions as shown in FIG. 17B. The longitudinal channels 381 are cut by advancing the end of the first mandrel 370 in the direction M into the distal end portion of the tube 2. As shown in FIG. 17B a small gap 378 is maintained between the distal end of the second mandrel 360 and the end of the first mandrel 370. At this stage an additional force is applied to the second mandrel 360 to cause the portion of the tube containing the gap 378 to collapse and bulge outward to fill the circular cavity 391 at the end of the third mandrel 390 as shown in FIG. 17C. The tube 2 is thereafter removed from the apparatus as shown in FIG. 17D.

Upon having produced the tube 2 having the longitudinal channels 382 and the enlarged external profile 398, the distal end portion of the tube 2 is subjected to one or more grinding processes as described above to form the bevel 6 of the needle as shown in FIG. 17E. As seen in FIG. 17E, the diameter D6 of the enlarged external profile 398 is greater than the diameter D3 of the remainder of the needle tube 2.

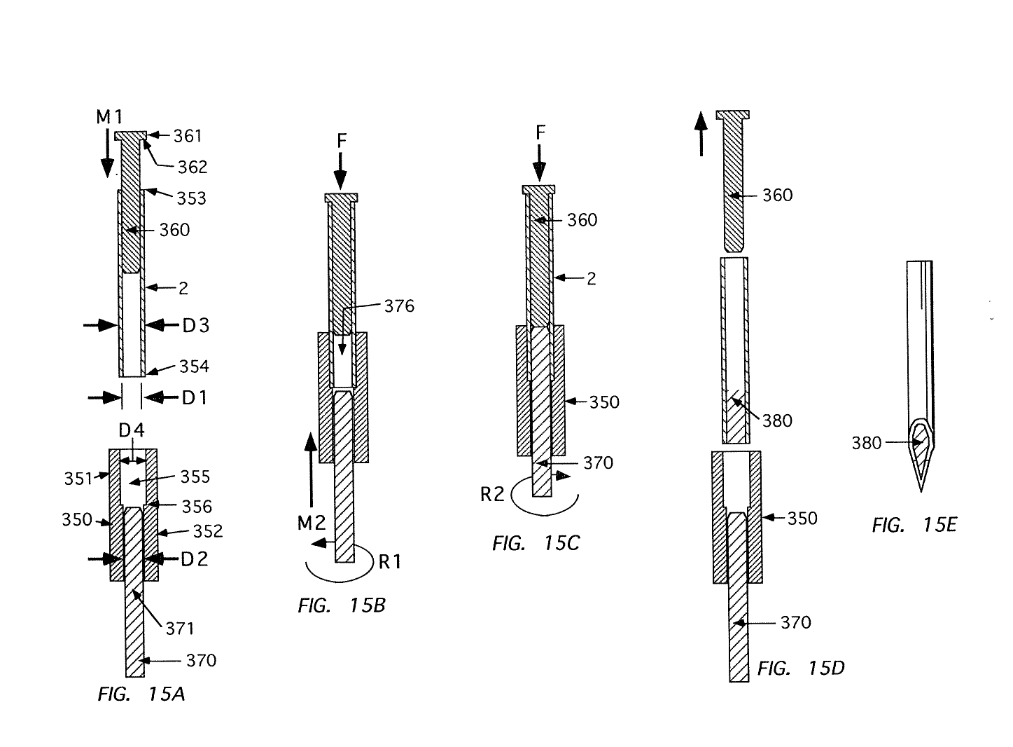

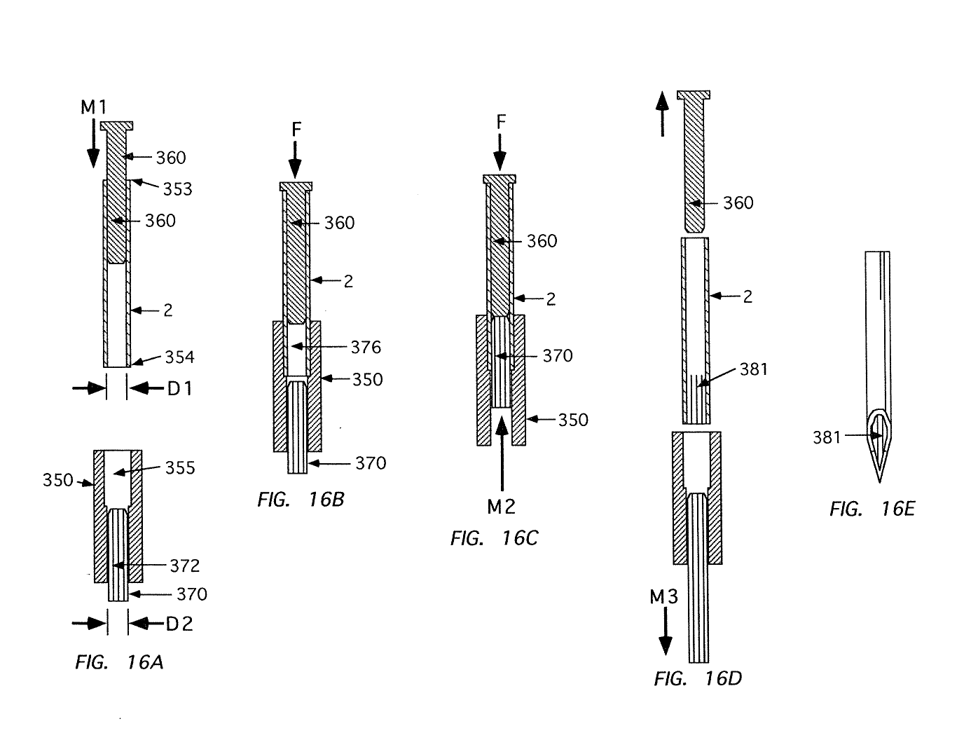

It is important to note that apparatus of FIGS. 17A-17D may be adapted with a first mandrel 371 similar in structure and function to that described in conjunction with FIGS. 15A-15D in order to produce a spiral channel rather than longitudinal channels. The same applies to the apparatus of FIGS. 18-20 discussed below which are directed to the formation of ultrasonic reflectors in the form of longitudinal channels.

FIGS. 18A-18E illustrate an apparatus and method of forming an ultrasonic reflector at the distal end of a tube 2, and to provide the tube 2 with an enlarged external profile at its distal end at the location of the ultrasonic reflector. The apparatus is similar to that of FIG. 16A except that the cavity 355 of the die 350 has an enlarged internal diameter D7 that is larger than the external diameter D3 of the tube 2. The apparatus also includes a third mandrel 400 that surrounds the tube 2. As shown in the figures, the tube 2 containing the second mandrel 360 slides within the third mandrel 400.

As shown in FIG. 18B, initially the first mandrel 370 is positioned within the second end of the die 350 and the distal end 354 of the tube is positioned within the enlarged cavity 355 of the die so that the distal end 354 rests on the ledge 356. The second mandrel 360 is also fully inserted into the tube 2 so that the flange surface 362 rests against the proximal end 353 of the tube 2. The tube 2 containing the second mandrel 360 is also fully inserted into the third mandrel 400. With the distal end 401 of the third mandrel 400 resting on the proximal end 359 of the die 350 forces F are applied to the second and third mandrels to maintain them in the positions shown in FIG. 18B. The longitudinal channels 381 are cut by advancing the end of the first mandrel 370 in the direction M1 into the distal end portion of the tube 2. As shown in FIG. 18C, the diameter D2 of the first mandrel 370 is sufficiently large to cause the distal end portion of the tube 2, into which the first mandrel is inserted, to expand outwardly until the outer wall 4 of the tube 2 contacts the inner wall of the enlarged diameter cavity 355. The tube 2 is thereafter removed from the apparatus as shown in FIG. 18D having an enlarged diameter distal end with longitudinal channels 381 formed therein. Thereafter, the distal end portion of the tube 2 is subjected to one or more grinding processes as described above to form the bevel 6 of the needle as shown in FIG. 18E. As seen in FIG. 18E, the resultant outer diameter D8 at the distal end of the tube is greater than the outer diameter D3 of the remainder of the needle tube 2.

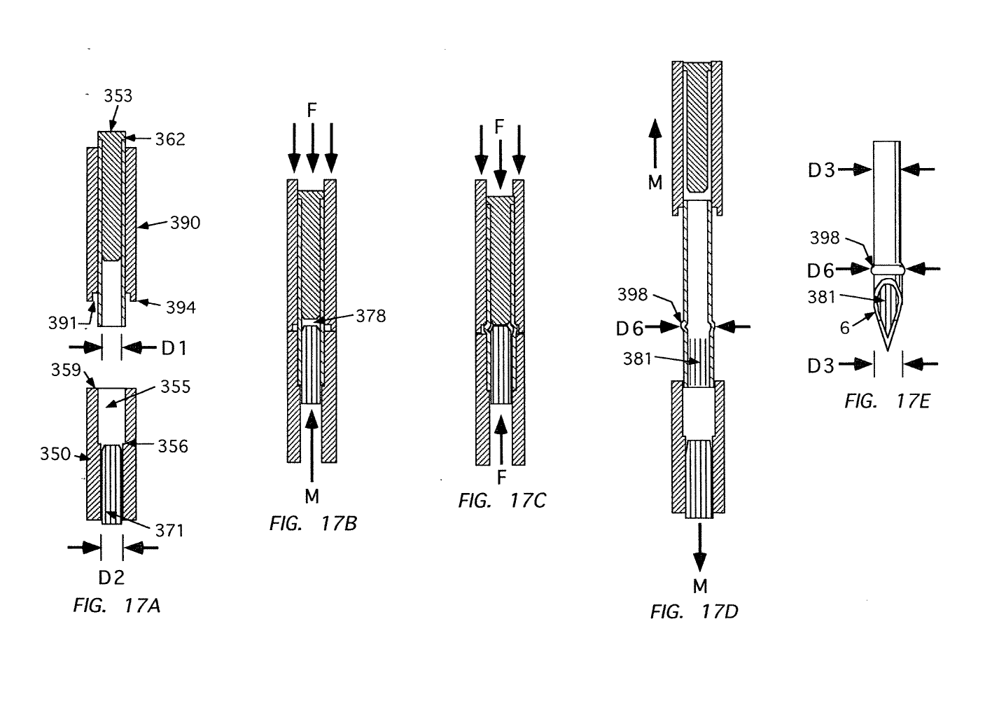

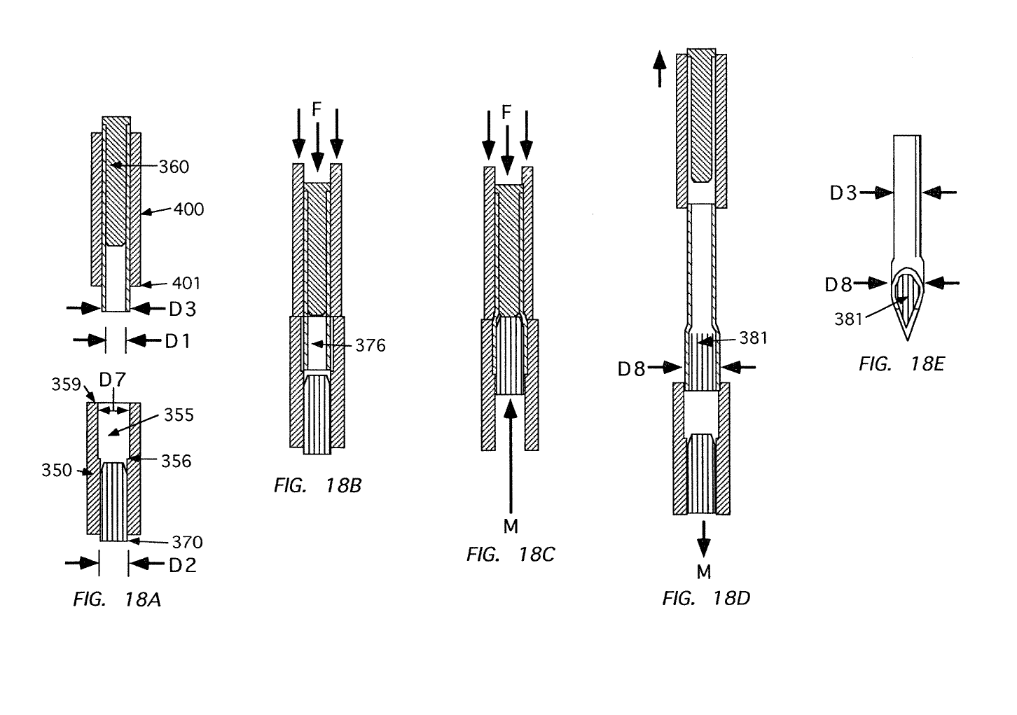

The apparatus and method of FIGS. 19A-19E are similar to those of FIGS. 16A-16E in that a first mandrel 370 having one or more cutting elements 372 is used to form one or more longitudinal channels 381 in the inner wall 5 of tube 2. The difference lies in the construction of the second mandrel identified by reference numeral 366 in FIG. 19A. Unlike the second mandrel 360 in the implementation of FIG. 16A, the second mandrel 366 in the present implementation has a length that is equal to or longer than the length of the tube 2. As a result, when the formation of the longitudinal channels 381 is complete, the second mandrel 366 may be advanced entirely through the through passage of the tube 2 to expel any debris that may have been created during the cutting process.

As shown in FIGS. 19A-19C, prior to and during the channel cutting process the proximal end 360 of the second mandrel 366 is maintained a distance away from the proximal end 353 of the tube 2. As such, the void 376 in the tube 2 is maintained during the cutting process. According to one implementation, the second mandrel 366 is maintained in this initial position by an elongate bushing 410 that is disposed between the proximal flange surface 362 of the second mandrel and the proximal end 353 of the tube 2. Upon the channels 381 having been formed and the first mandrel 370 removed from the tube 2, the bushing 410 is removed as shown in FIG. 19D and the second mandrel 366 is advanced to at least the distal end of the tube 2 for the purpose of removing debris created during the cutting process.

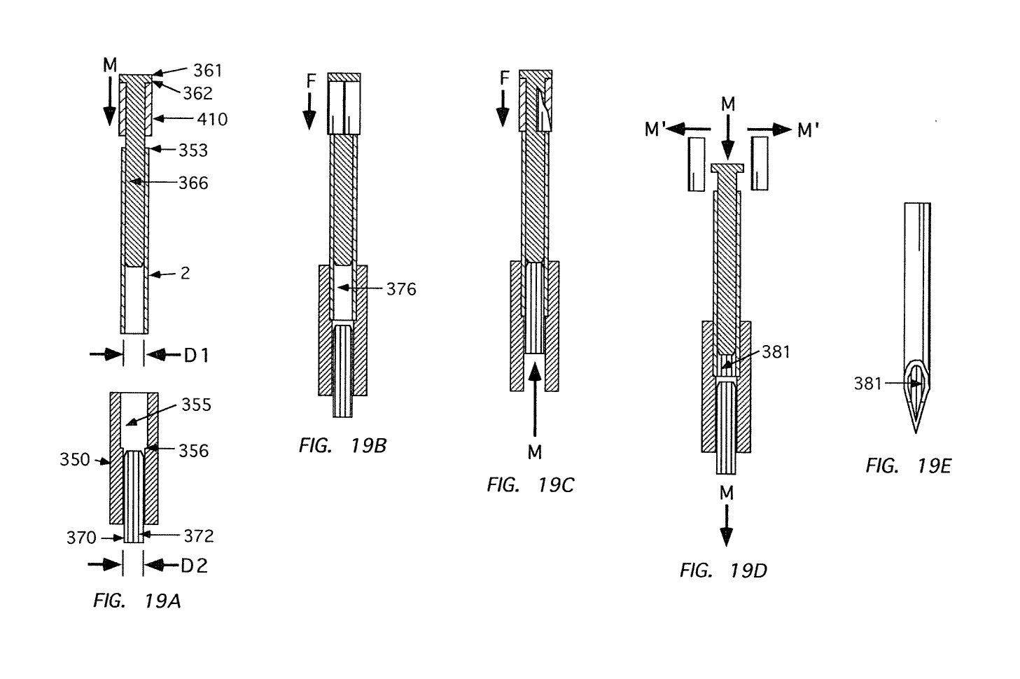

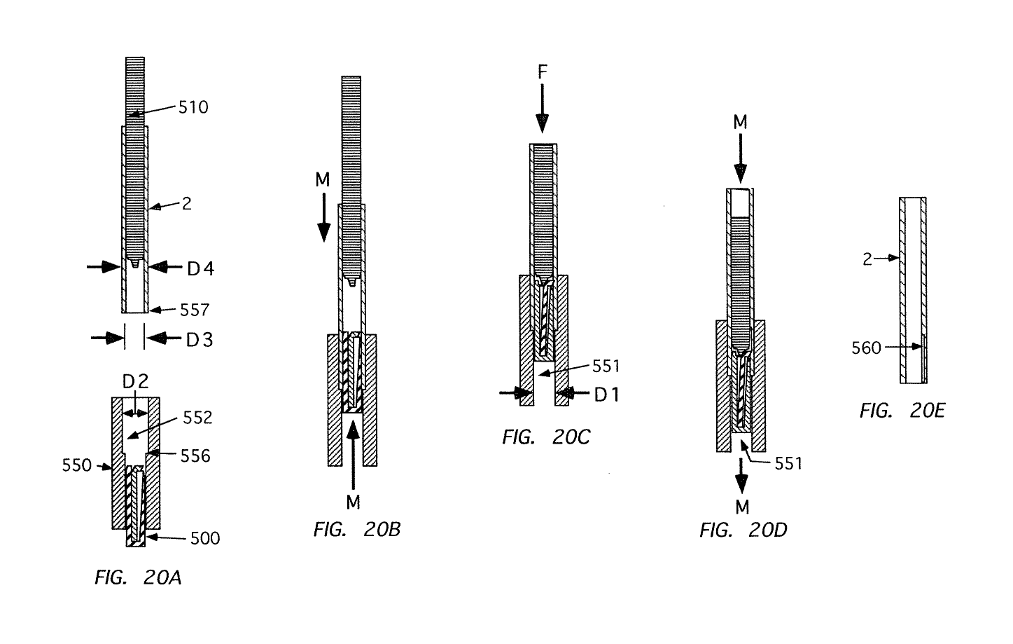

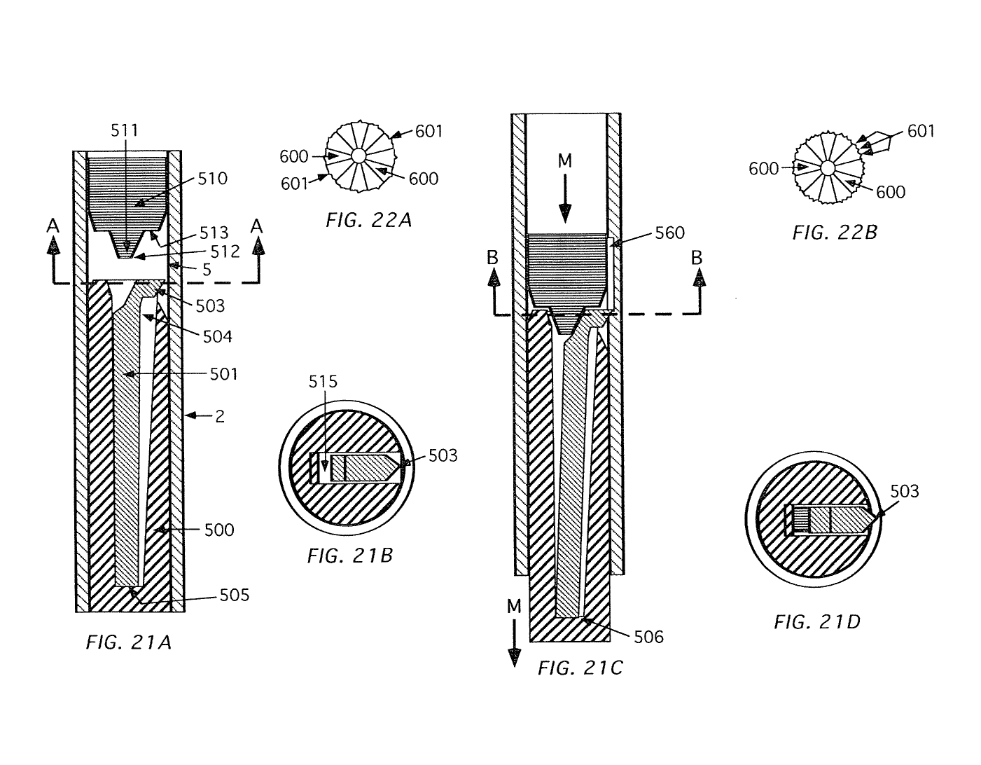

FIGS. 20 and 21 illustrate an apparatus and method for creating a longitudinal channel within the inner wall 5 of a tube 2. The apparatus includes a cutting assembly in the form of an elongate arm 501 with a blade 503 positioned at its distal end. The elongate arm 501 resides within a cavity 504 of a cylindrical housing 500 that is configured for being inserted into the distal end of the tube 2. The external diameter of the housing 500 is slightly smaller than the inner diameter of the tube 2, there being a sliding fit between the outer wall of the housing 500 and the inner wall 5 of the tube. The elongate arm 501 has a proximal end 505 that rests on the bottom wall 506 of the cavity 504. The elongate arm 501 and cavity 504 are constructed in a way that allows the elongate arm 501 to pivot within the cavity 504 so that the blade 503 is moveable between a non-cutting position as shown in FIGS. 20A, 20B, 21A and 21B, and a cutting position as shown in FIGS. 20C, 20D, 21C and 21D.

According to one implementation the position of the blade 503 is manipulated by the use of an elongate mandrel 510 that has a distal-most feature that is configured to act on the distal end of the elongate arm 501. In the implementation shown in the figures, the distal-most feature is a distally extending protrusion 511, the cross-section of the protrusion 511 at its tip 512 being smaller than the cross-section at its base 513. As most clearly shown in FIGS. 21A and 21B, when the blade 503 is in the non-cutting position a gap 515 exists between the backside of the blade 503 and the inner wall of the cavity 504 that is of a sufficient size to receive only the distal-most end portion of the protrusion 511. However, as the distal end of the mandrel 510 is further pushed against the backside of the blade 3, the blade is forced radially outward to cut into the wall 5 of the tube 2 as most clearly shown in FIGS. 21C and 21D. Continued advancement of the mandrel 510 into the tube 2 causes the housing 500 to move distally within the tube as shown in FIGS. 20C and 20D. During such movement the blade 503 maintains its cutting position to form a channel 560 in the wall 5 of the tube 2 as shown in FIGS. 20D. 20E and 21C.

According to the implementation of FIGS. 20A-20D a die 550 is used to facilitate the cutting process. The die 550 includes a first end having a first circular cavity 551 with a first inner diameter D1 and a second circular cavity 552 with a second inner diameter D2 that is greater than the first diameter D1. According to one implementation the inner diameter D3 of the tube is equal to the inner diameter D1 of the first circular cavity 551. The diameters D1 and D3 of the respective first circular cavity 551 and the tube 2 allow the housing 500 of the cutting assembly to be advanced therein with there being a sliding fit between them. The outer diameter D4 of the tube 2 is slightly smaller than the inner diameter D2 of the second cavity 552, with there preferably being a sliding fit between them.

As shown in FIG. 20A, the cutting assembly housing 500 is first introduced into the first circular cavity 551 of the die 550. The distal end of the tube 2 is then loaded into the second circular cavity 552 so that the distal end 557 of the tube rests on a ledge 556 located where the first and second circular cavities meet. The housing 500 is then inserted into the distal end of the tube 2 as shown in FIG. 20B so that the blade 3 is located at the proximal-most location of the channel to be cut. The mandrel 510 having been inserted into the open proximal end of the tube 2 is then advanced distally to cause the channel 560 to be cut into the wall 5 of the tube 2 in the manner described above.

The cutting assembly of FIGS. 20 and 21 may be modified to facilitate the simultaneous cutting of multiple channels by providing a plurality of cutting arms 600 as schematically illustrated in FIGS. 22A and 22B. In the implementation of FIG. 22A each cutting arm 600 includes a single blade 601. In the implementation of FIG. 22B each cutting arm 600 includes multiple blades 601.

In the forgoing description, ultrasonic reflectors positioned within the exposed area 17 of the bevel 2 of the needle have been disclosed. Attention will now been turned to the provision of ultrasonic reflectors on or within the grind surfaces 14, 15 and 16 of the bevel 6.

As explained above, the shape of the bevel 6 is generally produced by first grinding the distal end of the tube 2 to produce a sloped profile 9 as shown in FIG. 1B that runs from the proximal end of the bevel to the distal end of the tube 2. To create the lancet 7, a second set of grindings is generally made on the tube 2 to further reduce the tube profile to a point at tip 8. This may be accomplished, for example, by producing on each side of the lancet 7 a sloped profile 10 (as shown in FIG. 1B) that is distally co-extensive to and different than the sloped profile 9. FIG. 1C depicts a top view of the needle 1 that shows the top side of the bevel 6. Reference numeral 14 identifies a first grind surface produced during the first grinding of the tube 2. Reference numerals 15 and 16 identify second grind surfaces produced during the second set of grindings of the tube 2.

According to one implementation, as shown in FIG. 23, upon the completion of producing the grind surfaces 14, 15, and 16, one or more of the surfaces is roughened to increase the echogenicity of the one or more surfaces.

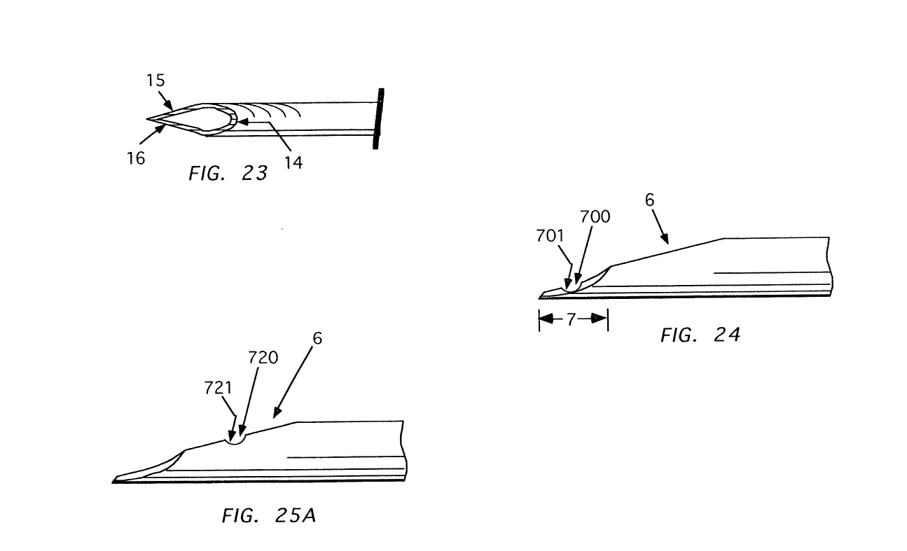

As seen in FIG. 24, an ultrasonic reflector 700 is formed in one or both of the grind surfaces 15 and 16. According to one implementation the ultrasonic reflector 700 comprises a curved indentation/notch having at least one proximal facing surface 701. According to one implementation the ultrasonic reflector 700 is formed by an additional grinding step that occurs after the formation of surfaces 14 and 15.

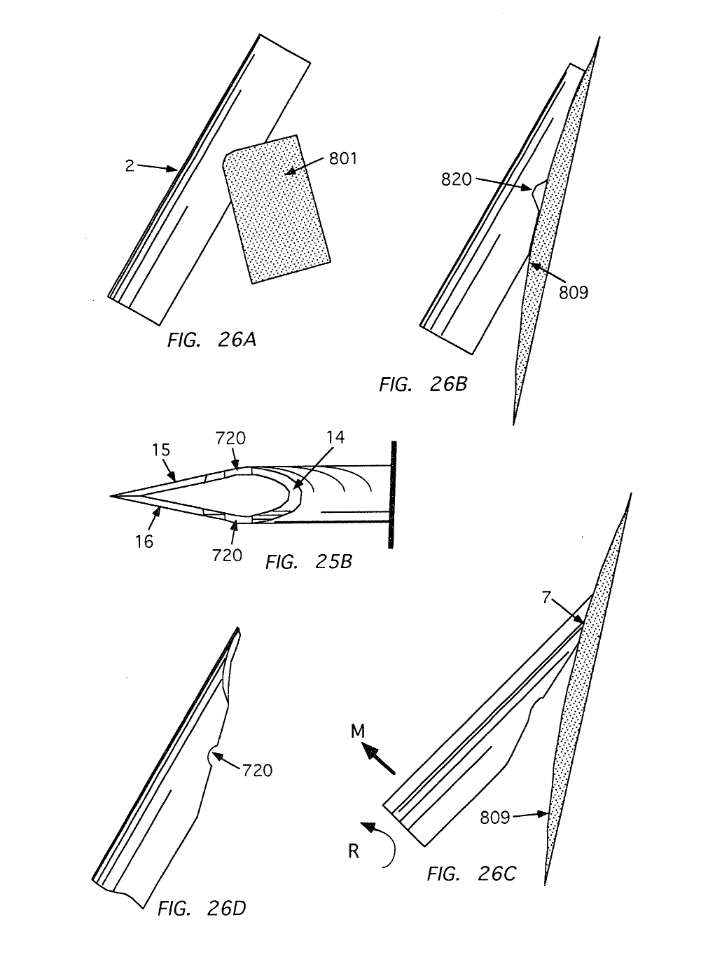

According to other implementations an ultrasonic reflector 720 is formed in the grind surface 14 as shown in FIGS. 25A and 25B. According to one implementation the ultrasonic reflector 720 comprises a curved indentation/notch having at least one proximal facing surface 721.

FIGS. 26A-26D illustrate a method of forming the ultrasonic reflector 720 according to one implementation. A first step, as shown in FIG. 26A involves grinding the external surface of the tube 2 with a first grind wheel 801 to form a curved notch 820 into the wall of the tube as shown. A second step, as shown in FIG. 26B involves subjecting the distal end of the tube 2, containing the previously formed curved notch 820, to a second grinding (using grind wheel 809) to produce the sloped profile 9 that runs from the proximal end of the bevel to the distal end of the tube 2. To create the lancet 7, a third set of grindings is made on the tube 2 to further reduce the tube profile to a point at tip 8. The tube 2 may be rotated R and moved M with respect to the grind wheel 809 to produce the resultant grind surfaces 14, 15 and 16 as shown in FIG. 25B and 26D.

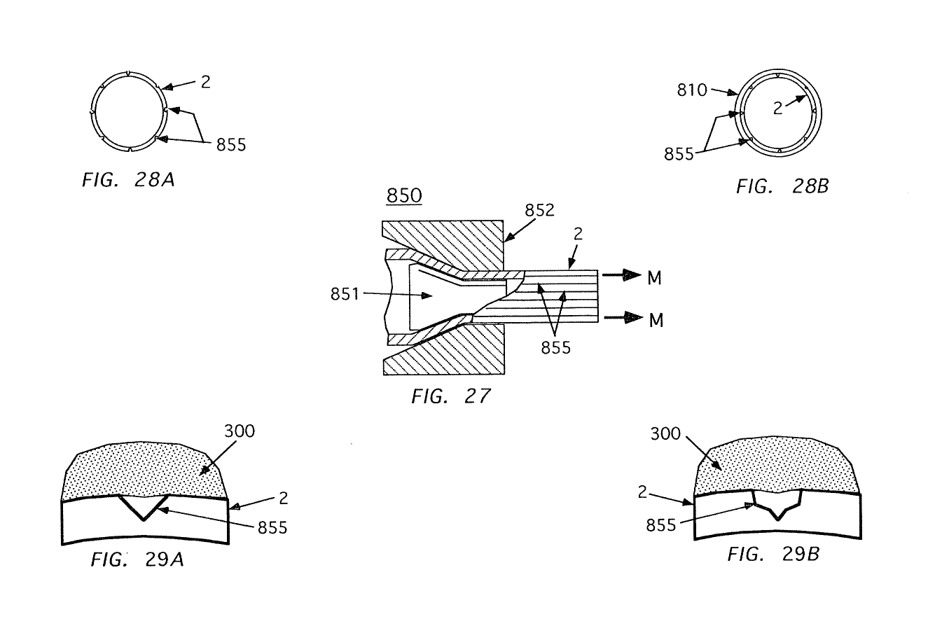

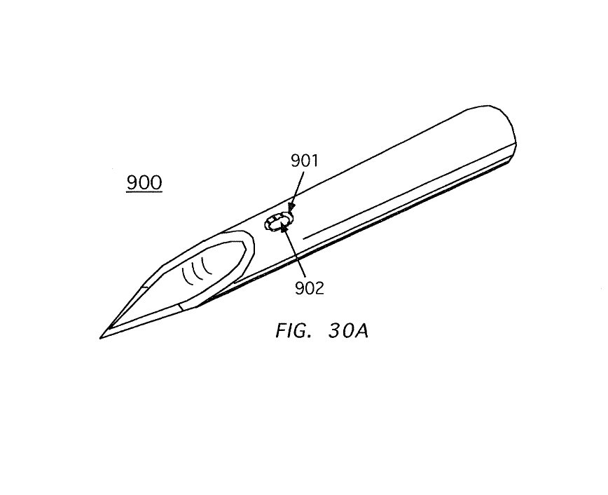

FIG. 27 shows a side view of an extrusion tool 850 that includes a mandrel 851 and a female die 852. In the implementation shown, the female die 852 includes cutting elements 153 (not visible in FIG. 27) that are dispersed equidistantly about the inner circumference of the die. According to one implementation, stainless steel tube stock is drawn through the extrusion tool 850 in the direction M in order to produce the needle tube 2. During the extrusion process the cutting elements impinge on the outer wall 4 of the tube to form a plurality of longitudinal channels 855 that run the length of the tube. FIG. 28A is a cutaway frontal view of the tube 2 with a partial cross-sectional portion showing the longitudinal channels 855. Upon having produced the tube 2 having the longitudinal channels 855, the distal end of the tube 2 is subjected to one or more grinding processes as described above to form the bevel 6 of the needle. FIGS. 29A and 29B show cross-sections of channels 855 according to some implementations. According to some implementations the tube 2 is rotated as it is advanced through the die 852 to produce one or more spiral channels in the outer wall 4 of the tube 2.

FIG. 28B is a cross-sectional view of the tube of FIG. 28A located inside the lumen of a catheter 810, such as an I.V. catheter.

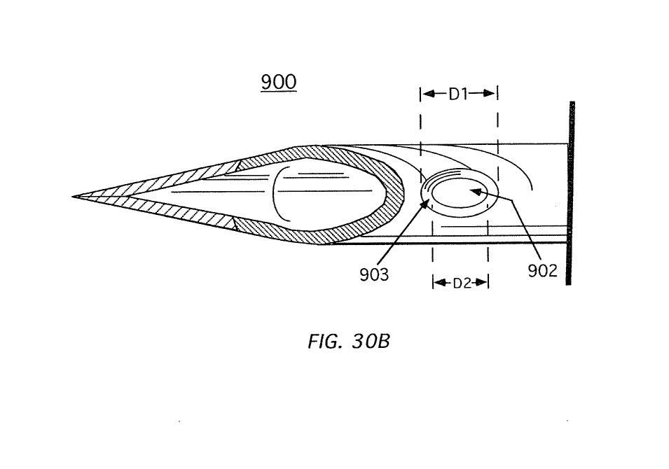

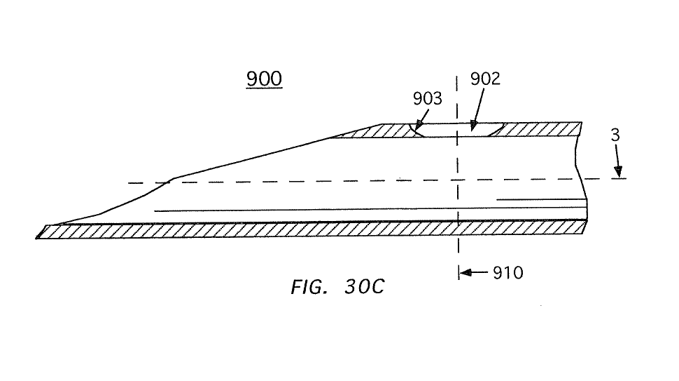

FIG. 30A is an isometric view of a medical needle 900 having an ultrasonic reflector 901 produced by forming an aperture 902 in the tube 2 that extends between the inner and outer walls 4 and 5 of the tube 2 at a location proximal to the bevel 6. FIG. 30B is a top view of the needle 90. FIG. 3C is a cross-sectional side view of the needle 90. The diameter D1 of the aperture 902 at the outer wall 5 of the tube 2 is greater than the diameter D2 of the aperture 902 at the inner wall 5 of the tube 2, with the inner circumferential wall 903 of the aperture 902 being sloped inward in a direction toward the radial center of the tube 2. This configuration has several advantages. First it provides a larger circumferential surface for reflecting ultrasonic waves directed toward the aperture. This, in conjunction with, the sloped surface of the inner circumferential wall 903 enhances the ultrasonic reflector's ability to reflect ultrasonic waves back toward an ultrasonic transceiver located above the top side of the needle amongst a wider range of angular orientations of the tube of the needle in comparison to an aperture having a uniform diameter throughout its length.

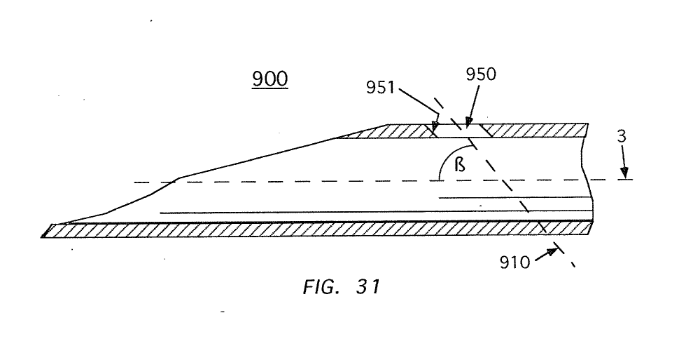

In the implementations of FIG. 30A-30C the central axis 910 of the aperture 902 is substantially perpendicular to the longitudinal axis 3 of the needle tube 2. However, according to other implementations, as shown in FIG. 31, a slanted aperture 950 is formed in the wall of the needle tube 2 so that the central axis 910 of the aperture 950 is not perpendicular to the longitudinal axis 3 of the tube 2. This configuration has several advantages. First it provides a larger exposed proximal facing surface 951 for reflecting distally directed ultrasonic waves. This, in conjunction with the sloped surface of the inner circumferential wall 952 enhances the ultrasonic reflector's ability to reflect ultrasonic waves back toward an ultrasonic transceiver located above the top side of the needle amongst a wider range of angular orientations of the tube of the needle in comparison to an aperture arranged orthogonal to the central axis of the needle with a uniform diameter throughout its length.

According to one method the slanted aperture 950 is formed by a tool or laser that bores through the wall of the tube 2 at an angle. According to one implementation the tool or laser is aligned centered with the top 13 of the tube, facing in the proximal direction. The slanted aperture is then cut at a target location on the outer wall 4 of the tube at a location proximal to the bevel 6.

According to some implementations the centerline of the central axis 910 of the slanted aperture 901 forms an angle β with respect to the central axis 3 of the tube in the range of between 20 to 80 degrees, and preferably between 30 to 70 degrees. However, in any event the central axis 910 of the slanted aperture 901 is never orthogonal to the central axis 3 of the tube 2.

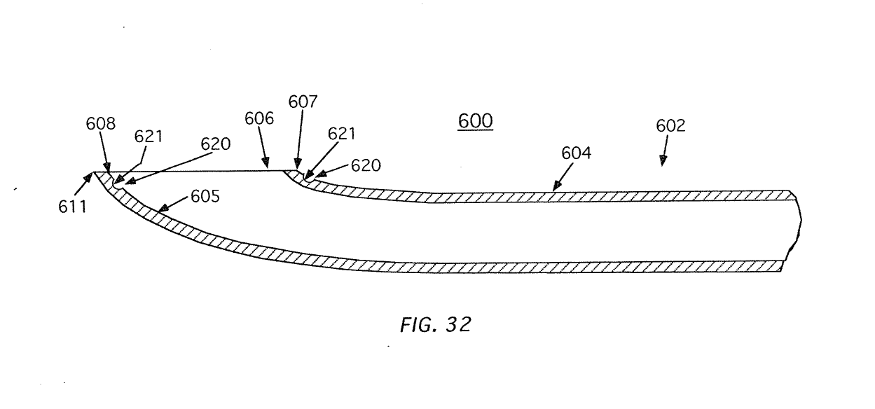

FIG. 32 illustrates a cross-sectional side view of an epidural need 600 according to one implementation. The needle comprises a tube 602 defined by a wall having an outer surface 604 and an inner surface 605. The epidural needle is distinguished by a change-in-axis that occurs in the longitudinal axis running through the tube 602 passageway. The distal end of the needle includes an opening 606 that has a proximal side 607 and a distal side 608, with the tip 611 of the needle residing on the distal side 608. According to one implementation, an ultrasonic reflector 621 in the form of a curved cavity, like that of cavity 21a above, that is provided in the inner wall of the tube 602 near the tip 611.

According to other implementations another ultrasonic reflector 620 is provided in lieu of or in combination with the reflector 621. According to one implementation, reflector 621 is in the form of a curved cavity, like that of cavity 21a above, that is provided on the outer wall of the tube 602, on the proximal side 607 of the opening 606, near the distal end of the tube.

The particular features, structures or characteristics of any implementation described above may be combined in any suitable manner, as would be apparent to one of ordinary skill in the art from this disclosure, in one or more implementations.

1-20. (canceled)

21. A method for providing unobstructed visualization of a distal tip of a medical needle inserted in a patient's tissue, said method comprising:
providing said needle as an elongate tube having a longitudinal central axis;
providing a distally tapered bevel extending lengthwise along a top side of the elongate tube and terminating at the distal tip such that the bevel exposes an exposed area on a tube inner wall surface extending lengthwise along the needle;
defining an ultrasound reflective contour in said exposed area viewable from said top side through said bevel;
transmitting ultrasound energy through the patient's tissue and through the bevel and onto the reflective contour; and
receiving ultrasound energy reflected from the ultrasound reflective contour.

22. The method of claim 21 wherein providing a distally tapered bevel comprises providing the exposed area diametrically opposite said first side along a bottom side of said elongate tube.

23. The method of claim 21 wherein defining an ultrasound reflective contour comprises defining a spiral channel in and lengthwise along the inner wall of the elongate tube.

24. The method of claim 23 wherein providing a distally tapered bevel includes defining a lancet at the distal end of the bevel, the spiral channel being disposed lengthwise only along the lancet.

25. The method of claim 23 wherein defining the spiral channel includes providing the spiral channel with a plurality of spaced-apart surfaces facing proximally of the elongate tube.

26. The method according to claim 25 further comprising defining the proximally facing surfaces as sloped surfaces.

27. The method according to claim 25 further comprising defining the proximally facing surface as curved surfaces.

28. The method according to claim 27, further comprising defining the curved surface with a semicircular shape.

29. The method according to claim 23, further comprising defining the spiral channel with a V-shape cross-section.

30. The method according to claim 23, further comprising defining the spiral channel with a U-shape cross-section.

31. The method according to claim 23 further comprising defining the spiral channel with a closed bottom and an open top, the channel having a height as measured between the closed bottom and open top, the open top having a length, the length to height ratio being equal to or greater than one.

32. The method according to claim 23 wherein defining the spiral channel includes providing the spiral channel with a plurality of spaced-apart surfaces facing proximally of the elongate tube such that:
at least one proximally facing surface has a base, and
the spiral channel includes a distally facing surface having a base that is spaced-apart from the base of the at least one proximally facing surface.

33. The method according to claim 23 wherein defining the spiral channel includes providing the spiral channel with a plurality of spaced-apart surfaces facing proximally of the elongate tube such that the proximally facing surfaces are sloped at an angle in relation to the longitudinal central axis in a range between 20° and 60°.

34. The method of claim 21 wherein defining the ultrasound reflective contour comprises defining a cavity in said exposed area having at least one surface portion facing toward a proximal end of the elongate tube.

35. The method of claim 34 wherein defining the cavity includes providing said at least one surface portion oriented oblique to an axis oriented orthogonal to the longitudinal central axis.

36. The method of claim 21 wherein defining the ultrasound reflective contour comprises defining a longitudinally extending channel in said exposed area.

37. The method of claim 36 wherein the longitudinally extending channel is defined with a V shape cross-section.

38. The method of claim 21 wherein defining the ultrasound reflective contour comprises defining a roughened surface that occupies at least a substantial portion of said exposed area.
